(19) United States
(12) Patent Application Publication
(10) Pub. No.: US 2026/0338761 A1
(43) Pub. Date: Oct. 29, 2026

(54) FLEXIBLE ELECTRICAL CONTROL OF NANOPORE ARRAYS

(71) Applicant: ILLUMINA, INC., San Diego, CA (US)

(72) Inventors: Kirkpatrick Norton, San Diego, CA (US); Jeremy Ford, San Diego, CA (US); Rico Otto, San Diego, CA (US); Boyan Boyanov, San Diego, CA (US); Rean Musa, San Diego, CA (US)

(21) Appl. No.: 19/656,398

(22) Filed: Apr. 23, 2026

Related U.S. Application Data
(60) Provisional application No. 63/794,607, filed on Apr. 25, 2025.

(51) Int. Cl. G01N 33/487

(52) U.S. Cl. CPC G01N 33/48721

(57) ABSTRACT

A nanopore integrated circuit can include a semiconductor substrate; a plurality of nanopore cells formed over the substrate, each nanopore cell having a cis electrode connection and a trans electrode, wherein the plurality of nanopore cells are grouped into a plurality of nanopore pixel groups; drive circuitry including one or more switching devices formed on the substrate and coupled to the trans electrodes of the plurality of nanopore cells that: delivers stimulus signals to the trans electrodes of the plurality of nanopore cells, wherein the stimulus signals are delivered to the plurality of nanopore pixel groups in a rolling stimulus operation; and measures output signals of the plurality of nanopore pixel groups in a rolling shutter operation, wherein an output signal of a particular nanopore cell is produced by the nanopore cell responsive to a stimulus signal and corresponds to an analyte translocated with respect to the particular nanopore cell.

### TECHNICAL FIELD

Embodiments of the present disclosure relate to nanopore ASICs including nanopore structures, stimulus and readout circuits and topologies, and techniques for stimulating nanopores and groups of nanopores.

### BACKGROUND

DNA sequencing techniques may involve performing a large number of controlled reactions on support surfaces or within predefined reaction chambers. The controlled reactions may then be observed or detected, and subsequent analysis may help identify properties of the polynucleotide involved in the reaction. Examples of such sequencing techniques include next-generation sequencing (NGS) or massive parallel sequencing involving sequencing-by-ligation, sequencing-by-synthesis, reversible terminator chemistry, or pyrosequencing approaches.

Certain NGS technologies utilize a nanopore, which can provide a path for an ionic electrical current across a membrane. In one example, disruptions in the electrical current can be used to characterize features of a nucleic acid within a passage of the nanopore. For example, as an oligonucleotide traverses through the nanopore, the polynucleotide influences the electrical current through the nanopore. Nanopore sequencing may include the use of processive enzymes such as polymerases or helicases to aid the translocation of oligonucleotides through the nanopore. Each passing nucleotide, or series of nucleotides, that passes through the nanopore yields a characteristic current disruption or blockage current. These characteristic blockage currents of the traversing oligonucleotide are detected and used to determine the sequence of the oligonucleotide.

In some applications, a plurality of nanopores, such as an array of nanopores, can be formed on a semiconductor substrate as part of an application specific integrated circuit (ASIC) that can be incorporated into devices or systems for DNA sequencing.

### SUMMARY

Disclosed herein are devices and techniques for DNA sequencing. This can include providing a template comprising nucleic acids of the target polynucleotide, hybridizing a primer to the template, extending the primer to form a complementary strand of the template. At least a portion of the complementary strand can be disposed through a nanopore, followed by detecting a signal as a result of the disposing, and generating sequence data of the target polynucleotide from the signal. The nanopore may be disposed between a cis electrode, a trans electrode, and an aperture extending through the cis side and trans side, such that the complementary strand translocates with respect the nanopore. The detected signal can be an electrical signal associated with the nanopore while the complementary strand is translocated with respect to the nanopore.

A nanopore integrated circuit can include a semiconductor substrate; a plurality of nanopore cells formed over the substrate, each nanopore cell having a cis electrode connection and a trans electrode, wherein the plurality of nanopore cells are grouped into a plurality of nanopore pixel groups; drive circuitry including one or more switching devices formed on the substrate and coupled to the trans electrodes of the plurality of nanopore cells that: delivers stimulus signals to the trans electrodes of the plurality of nanopore cells, wherein the stimulus signals are delivered to the plurality of nanopore pixel groups in a rolling stimulus operation; and measures output signals of the plurality of nanopore pixel groups in a rolling shutter operation, wherein an output signal of a particular nanopore cell is produced by the nanopore cell in response to a stimulus signal and corresponds to an analyte translocated with respect to the particular nanopore cell.

The stimulus signals can include stimulus pulses of alternating polarities. The output signals can be measured using a bi-phase measurement corresponding to the stimulus pulses of alternating polarities. The stimulus signals delivered to adjacent pixel groups can include positive stimulus pulses delivered to a first subset of the adjacent pixel groups and negative stimulus pulses delivered to a second subset of the adjacent pixel groups. The drive circuitry can deliver input stimulus signals during a crosstalk and settling time interval and can measure the output signals during a quiet interval following the crosstalk and settling time interval. At least one of the cis electrode and the trans electrodes can be a non-Faradaic electrode. The drive circuitry can be further configured to deliver one or more electrical signals distinct from the stimulus signals to the plurality of nanopore cells to perform additional nanopore unit cell operations. The nanopore integrated circuit can further include one or more analog to digital converters for measuring the output signals of the plurality of nanopore pixel groups; and the rolling shutter operation measuring output signals of the plurality of nanopore pixel groups can be coordinated with a readout timing of the one or more analog to digital converters.

A method of driving a nanopore integrated circuit having a plurality of nanopore cells grouped into a plurality of nanopore pixel groups, each nanopore cell having a cis electrode connection and a trans electrode, can include: delivering stimulus signals to the trans electrodes of the plurality of nanopore cells, wherein the stimulus signals are delivered to the plurality of nanopore pixel groups in a rolling stimulus operation; and measuring output signals of the plurality of nanopore pixel groups in a rolling shutter operation, wherein an output signal of a particular nanopore cell is produced by the nanopore cell in response to a stimulus signal and corresponds to an analyte translocated with respect to the particular nanopore cell.

The stimulus signals can include stimulus pulses of alternating polarities. The method can further include measuring the output signal using a bi-phase measurement corresponding to the stimulus pulses of alternating polarities. The stimulus signals delivered to adjacent pixel groups can include positive stimulus pulses delivered to a first subset of the adjacent pixel groups and negative stimulus pulses delivered to a second subset of the adjacent pixel groups. Delivering stimulus signals to the trans electrodes of the plurality of nanopore cells can occur during a crosstalk and settling time interval; and measuring the output signals can occur during a quiet interval following the crosstalk and settling time interval. The method can further include delivering one or more electrical signals distinct from the stimulus signals to the plurality of nanopore cells to perform additional nanopore unit cell operations. The nanopore integrated circuit can include one or more analog to digital converters for measuring the output signals of the plurality of nanopore pixel groups, and the method can further include coordinating the rolling shutter operation measuring output signals of the plurality of nanopore pixel groups with a readout timing of the one or more analog to digital converters.

A nanopore integrated circuit can include a semiconductor substrate; a plurality of nanopore cells formed over the substrate, each nanopore cell having a cis electrode connection and a trans electrode, wherein the plurality of nanopore cells are grouped into a plurality of nanopore pixel groups; drive circuitry including one or more switching devices formed on the substrate and coupled to the trans electrodes of the plurality of nanopore cells that: delivers stimulus signals to the trans electrodes of one or more nanopore pixel groups; and performs a rolling shutter operation on the one or more nanopore pixel groups by selectively measuring output signals at the trans electrodes of nanopore cells of the one or more pixel groups, the output signals being produced by the nanopore cells in response to the stimulus signals, wherein an output signal of a particular nanopore cell corresponds to an analyte translocated with respect to the particular nanopore cell.

The stimulus signals can include stimulus pulses of alternating polarities. The output signals can be measured using a bi-phase measurement corresponding to the stimulus pulses of alternating polarities. The stimulus signals delivered to adjacent pixel groups can include positive stimulus pulses delivered to a first subset of the adjacent pixel groups and negative stimulus pulses delivered to a second subset of the adjacent pixel groups. The drive circuitry can deliver input stimulus signals during a crosstalk and settling time interval and can measure the output signals during a quiet interval following the crosstalk and settling time interval. The nanopore integrated circuit can further include one or more analog to digital converters for measuring the output signals of the plurality of nanopore pixel groups; and the rolling shutter operation measuring output signals of the plurality of nanopore pixel groups is coordinated with a readout timing of the one or more analog to digital converters.

A method of driving a nanopore integrated circuit having a plurality of nanopore cells grouped into a plurality of nanopore pixel groups, each nanopore cell having a cis electrode connection and a trans electrode, can include: delivering stimulus signals to the trans electrodes of one or more nanopore pixel groups; and performing a rolling shutter operation on the one or more nanopore pixel groups by selectively measuring output signals at the trans electrodes of nanopore cells of the one or more pixel groups, the output signals being produced by the nanopore cells in response to the stimulus signals, wherein an output signal of a particular nanopore cell corresponds to an analyte translocated with respect to the particular nanopore cell.

The stimulus signals can include stimulus pulses of alternating polarities. The method can further include measuring the output signals using a bi-phase measurement corresponding to the stimulus pulses of alternating polarities. The stimulus signals delivered to adjacent pixel groups can include positive stimulus pulses delivered to a first subset of the adjacent pixel groups and negative stimulus pulses delivered to a second subset of the adjacent pixel groups. Delivering stimulus signals to the trans electrodes of the plurality of nanopore cells can occur during a crosstalk and settling time interval; and measuring the output signals can occur during a quiet interval following the crosstalk and settling time interval. The nanopore integrated circuit can include one or more analog to digital converters for measuring the output signals of the plurality of nanopore pixel groups, and the method can further include coordinating the rolling shutter operation measuring output signals of the plurality of nanopore pixel groups with a readout timing of the one or more analog to digital converters.

A nanopore integrated circuit can include a semiconductor substrate; a plurality of nanopore cells formed over the substrate, each nanopore cell having a cis electrode connection and a trans electrode, wherein the plurality of nanopore cells are grouped into a plurality of nanopore pixel groups; drive circuitry including one or more switching devices formed on the substrate and coupled to the trans electrodes of the plurality of nanopore cells that: performs a rolling stimulus operation to deliver stimulus signals to the trans electrodes of the plurality of nanopore cells, wherein the stimulus signals are delivered to one or more nanopore pixel groups; and measures output signals of the one or more nanopore pixel groups, wherein an output signal of a particular nanopore cell is produced by the nanopore cell in response to a stimulus signal and corresponds to an analyte translocated with respect to the particular nanopore cell.

The stimulus signals can include stimulus pulses of alternating polarities. The output signals can be measured using a bi-phase measurement corresponding to the stimulus pulses of alternating polarities. The stimulus signals delivered to adjacent pixel groups can include positive stimulus pulses delivered to a first subset of the adjacent pixel groups and negative stimulus pulses delivered to a second subset of the adjacent pixel groups. The drive circuitry can deliver input stimulus signals during a crosstalk and settling time interval and can measure the output signals during a quiet interval following the crosstalk and settling time interval.

A method of driving a nanopore integrated circuit having a plurality of nanopore cells grouped into a plurality of nanopore pixel groups, each nanopore cell having a cis electrode connection and a trans electrode, can include: performing a rolling stimulus operation to deliver stimulus signals to the trans electrodes of one or more nanopore pixel groups; and measuring output signals of the one or more of nanopore pixel groups, wherein an output signal of a particular nanopore cell is produced by the nanopore cell in response to a stimulus signal and corresponds to an analyte translocated with respect to the particular nanopore cell.

The stimulus signals can include stimulus pulses of alternating polarities. The method can further include measuring the output signals using a bi-phase measurement corresponding to the stimulus pulses of alternating polarities. The stimulus signals delivered to adjacent pixel groups can include positive stimulus pulses delivered to a first subset of the adjacent pixel groups and negative stimulus pulses delivered to a second subset of the adjacent pixel groups. Delivering stimulus signals to the trans electrodes of the plurality of nanopore cells can occur during a crosstalk and settling time interval; and measuring the output signals can occur during a quiet interval following the crosstalk and settling time interval.

### BRIEF DESCRIPTION OF THE DRAWINGS

These and other features, aspects, and advantages of the disclosed embodiments will become better understood when the following detailed description is read with reference to the accompanying drawings in which like characters represent like parts throughout the drawings.

FIGS. 1A-C are schematic illustrations of certain embodiments of a nanopore flow cell.

FIGS. 2 is a schematic illustration of certain embodiments of a nanopore sequencing system.

FIGS. 3A-C are schematic illustrations of certain embodiments of preparing and sequencing of an elongated polymer.

FIG. 4 depicts a diagram of a nanopore and an equivalent electrical circuit.

FIG. 5 depicts a simplified schematic of a plurality of nanopores.

FIG. 6 depicts a schematic of a universal nanopore pixel circuit for global shutter or rolling shutter operation.

FIG. 7 depicts a rolling shutter pixel circuit for rolling shutter operation.

FIG. 8 depicts an example pixel stimulus technique for a nanopore.

FIG. 9A depicts a rolling shutter stimulus and sampling operation for groups of nanopore pixels.

FIG. 9B depicts a global shutter stimulus and sampling operation for nanopore pixels.

FIG. 10 depicts pore resistance and output voltage plots for an example nanopore depicting long time constant hysteresis/memory effects.

FIG. 11 depicts a bi-phase readout technique for mitigating at least some effects of the long time constant hysteresis/memory effects described above.

FIG. 12 depicts pore resistance and output voltage plots for an example nanopore employing biphase readout to depict at least partial mitigation of long time constant hysteresis/memory effects.

FIG. 13 is a simplified schematic of a nanopore integrated circuit with multiple nanopore groups driven from the ADC side/trans electrode.

FIG. 14 is a simplified schematic of a nanopore integrated circuit with multiple nanopore groups driven from the ADC side/trans electrode.

FIG. 15 illustrates various operating waveforms of a nanopore driven from the ADC side/trans electrode together with additional details of the drive circuitry.

FIG. 16 illustrates a schematic diagram of circuitry for driving and reading separate groups of nanopores driven from the ADC side/trans electrode using a rolling shutter.

FIG. 17 illustrates various operating waveforms of a nanopore driven from the ADC side/trans electrode together with additional details of the drive circuitry.

The schematic drawings are not necessarily to scale. Like numbers used in the figures refer to like components, steps and the like. However, it will be understood that the use of a number to refer to a component in a given figure is not intended to limit the component in another figure labeled with the same or a similar number. In addition, the use of different numbers to refer to components is not intended to indicate that the different numbered components cannot be the same or similar to other numbered components.

### DETAILED DESCRIPTION

The following discussion is presented to enable any person skilled in the art to make and use the technology disclosed and is provided in the context of a particular application and its requirements. Various modifications to the disclosed implementations will be readily apparent to those skilled in the art, and the general principles defined herein may be applied to other implementations and applications without departing from the spirit and scope of the technology disclosed. Thus, the technology disclosed is not intended to be limited to the implementations shown but is to be accorded the widest scope consistent with the principles and features disclosed herein.

### Nanopore Sequencing Introduction

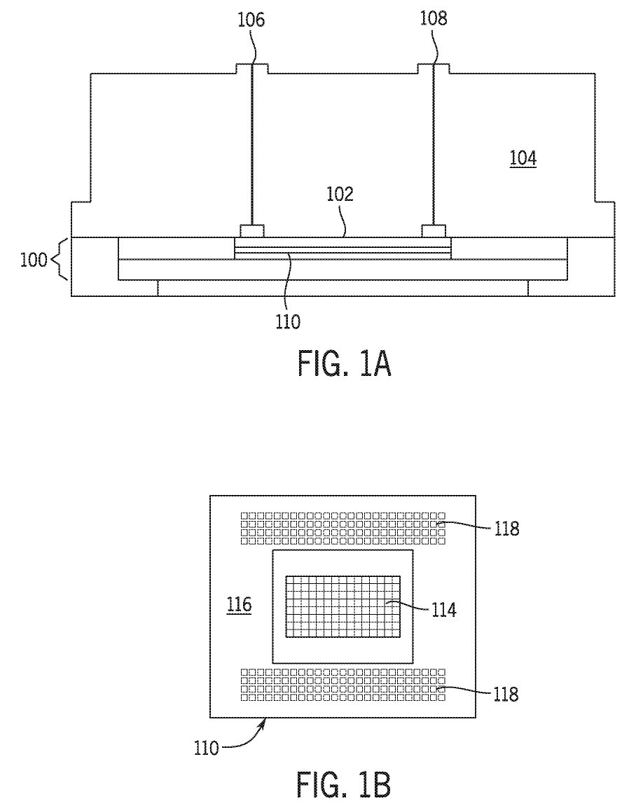

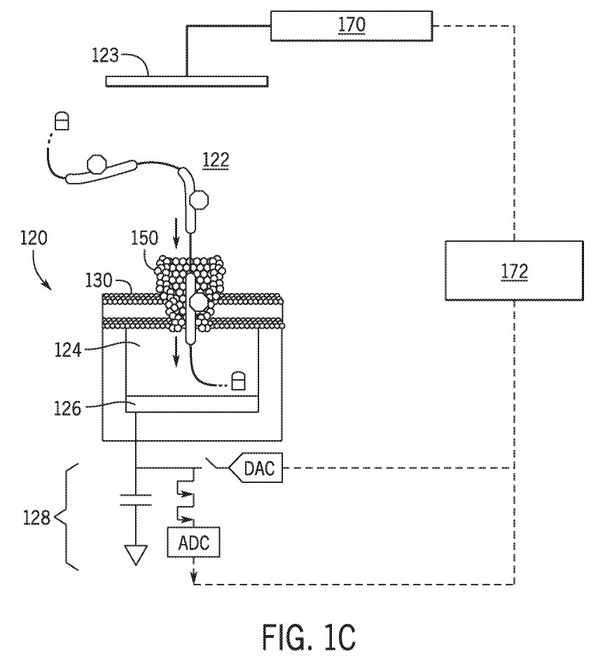

FIGS. 1A-C are schematic illustrations of certain embodiments of a nanopore flow cell 100. FIG. 1A is a cross-sectional schematic view of certain embodiments of the nanopore flow cell 100 comprising one or more integrated circuits 110 comprising an array of nanopore sensors or nanopore unit cells. The integrated circuit 110 and a lid 102 define one or more fluidic channels and define an array of cis/trans wells therebetween. In certain embodiments, the nanopore flow cell 100 may be housed in a cartridge 104 to be inserted into a sequencing instrument, library preparation station, other liquid handling system, and/or combinations thereof. The cartridge 104 includes one or more inlets 106 and one or more outlets 108 for delivery and removal of one or more fluids (e.g., liquid or gas) to the fluidic channel and to the array of cis/trans wells. For example, the one or more fluids and membrane components may be delivered to the array for formation of membranes over the trans wells. For example, the one or more fluids and polypeptide nanopore components may be delivered to the array for formation and insertion of polypeptide nanopores into the membranes. For example, the one or more fluids and analytes may be delivered to the array for capture and translocation of analytes through the nanopores. For example, the one or more fluids and other compounds may be delivered to the array for the compounds to participate in capture and/or translocation of analytes through nanopores. The nanopore flow cell 100 may further comprise structures between the integrated circuit assembly 110 and the lid 102 to define the array into one or more lanes, such as straight lanes and/or serpentine lanes. A plurality of inlets 106 and/or a plurality of outlets 108 may be individually addressable to allow for individual loading of the lanes.

FIG. 1B is a plan schematic view of certain embodiments of the integrated circuit 110 of FIG. 1A. The integrated circuit 110 circuit chips has an array 114 of nanopore sensors or nanopore unit cells. The integrated circuit 110 may be mounted on a printed circuit board 116 for coupling the integrated circuit 110 to electrical connectors 118, such as pogo pin connectors or other suitable connectors, for connection to a controller for operation of the nanopore sensor or nanopore unit cells.

FIG. 1C is a cross-section schematic view of certain embodiments of an individual nanopore sensor or nanopore unit cell defined by the array 114 of the integrated circuit 110 of FIGS. 1A-B and by the lid 102 of the flow cell 100. Each nanopore unit cell 120 comprises a cis well 122 and a trans well 124. For example, as shown in FIG. 1C, all or a group of nanopore unit cells may be associated with a common cis well with individual trans wells. In certain embodiments, the array 114 may comprise 1,000 or more, 10,000 or more, 100,000 or more, 1 million or more, 5 million or more, or 10 million or more nanopore unit cells. The layout of the array 114 may be rectilinear (i.e., rectangular), hexagonal, triangular, and other suitable layouts. The layout may also or alternatively be characterized in terms of the pitch, i.e., the spacing from the center of a trans well 124 to the center of an adjacent trans well 124 (center-to-center spacing). In certain embodiments, the pitch may by about 500 µm or less, about 100 µm or less, about 50 µm or less, about 30 µm or less, about 15 µm or less, or about 10 µm or less. In certain embodiments, the trans wells 124 may have an opening with a diameter of about 100 µm or less, about 50 µm or less, about 30 µm or less, about 15 µm or less, about 10 µm or less, or about 5 µm or less. In certain embodiments, the trans wells 125 may have an aspect ratio in a range from between about 10:1 and 1:10, between about 5:1 and 1:5, about 3:1 and about 1:3, about 2:1 and 1:2.

Each of the trans 124 wells may be associated with an individual trans electrode 126. As shown in FIG. 1C, a waveform generator 170 is coupled to the cis electrode 170 to apply a bias across the nanopore unit cell 170. In other embodiments, a waveform generator 170 may be coupled to the trans electrode or both the cis electrode and trans electrode. A controller 170 is coupled to waveform generator 170 and circuitry 128 to control the state of the nanopore unit cell 120.

Any appropriate voltage bias may be applied during membrane formation, pore insertion, analyte capture, analyte translocation, analyte sensing, analyte ejection, membrane destruction, or other nanopore unit cell operations. For example, in some embodiments, the voltage bias across the nanopore 150 provided by the waveform generator is from about -1 V to about +1 V, such as during analyte sensing or other operations.

The trans electrode 126 may be a non-faradaic electrode. Non-faradaic electrodes do not participate in a redox reaction in the nanopore unit cell. Examples of non-faradic electrodes include titanium nitride (TiN), tantalum nitride (TaN), and other suitable electrodes. Non faradaic electrodes include any suitable conductive materials, including electrochemically inert materials and electrochemically active materials, when operated under bias and buffer conditions that suppress Faradaic charge transfer reactions. The trans wells 124 may be further associated with circuity 128 of the integrated circuit 110, such as amplifiers, switches, capacitors, transistors, registers, digital-to-analog converters (DACs), analog-to-digital converter (ADCs), and other circuitry to control the state of the nanopore cells and/or to sense the conditions of the nanopore cell 120. For example, the circuitry 128 may also include amplifier(s) to amplify electrical signals passing through respective nanopores 20 associated with trans electrodes 22 that are addressed. In some embodiments, each nanopore unit cell 120 may be individually addressed or accessed by circuity 128.

The surface of the integrated circuit is patterned to define the trans wells 124. For example, the surface of the integrated circuit 110 may comprise a dielectric layer, such as silicon oxide or other suitable material, which is patterned to define the trans wells 124. Each trans well 124 may have an individual membrane 130 formed or painted thereover.

As used herein, a “membrane” is intended to mean a structure that normally inhibits passage of ions and/or molecules from one side of the membrane to the other side of the membrane. The molecules for which passage is inhibited can include, for example, nucleotides, nucleotide constructs, amino acids, amino acid constructs, and other compounds. However, if a nanopore is inserted into a membrane, then the channel of the nanopore may permit passage of molecules from one side of the membrane to the other side of the membrane.

Membranes suitable for use in the examples disclosed herein include amphiphilic polymer layers, including those of biological origin, such as amphiphilic lipid bilayers, or those of synthetic origin, such as amphiphilic polymer layer(s). Suitable amphiphilic polymer layers comprise one or more layers of amphiphilic block co-polymer layers. For example, a diblock AB copolymer may form a bilayer membrane. For example, a triblock ABA copolymer may form a monolayer and or a bilayer membrane. Examples of amphiphilic block co-polymer membranes, formation of membranes, and painting of membranes are disclosed in PCT International Patent Application Publication WO 2023/187111 A1, entitled “Barriers Including Biological Nanopore for DNA Sequencing, the Barriers Being Made Of Co-Polymers with End and/or Middle Groups, and Methods of Making the Same”, filed by Illumina Cambridge Limited (Cambridge, United Kingdom), published on Oct. 5, 2023, which is incorporated by reference in its entirety.

In other embodiments, the membrane may comprise one or more solid-state material layers formed using semiconductor processes, such as formed through the deposition of a dielectric layer or a combination of dielectric layers, such as silicon oxides, silicon nitride, silicon oxynitride, titanium oxide, tantalum oxide, hafnium oxide, aluminum oxide, and other suitable dielectric layers.

As shown in FIG. 1C, a biological nanopore 150 is inserted in membrane 130. Biological nanopores include, for example, polypeptide nanopores and polynucleotide nanopores. A “polypeptide nanopore” is intended to mean a nanopore that is made from one or more polypeptides. The one or more polypeptides can include a monomer, a homopolymer or a heteropolymer. Structures of polypeptide nanopores include, for example, an α-helix bundle nanopore and a β-barrel nanopore as well as all others well known in the art. Example polypeptide nanopores include α-hemolysin, Mycobacterium smegmatis porin A (MspA), aerolysin, gramicidin A, maltoporin, OmpF, OmpC, PhoE, Tsx, F-pilus, SP1, mitochondrial porin (VDAC), Tom40, outer membrane phospholipase A, CsgG, CsgG-CsgF complex, and Neisseria autotransporter lipoprotein (NaIP). Mycobacterium smegmatis porin A (MspA) is a membrane porin produced by Mycobacteria, allowing hydrophilic molecules to enter the bacterium. MspA forms a tightly interconnected octamer and transmembrane beta-barrel that resembles a goblet and includes a central constriction. Examples of α-hemolysin protein pores are disclosed in PCT International Patent Application Publication WO2007/057668 A1, entitled “Methods Using Pores”, filed by ISIS Innovation Limited (Oxford, United Kingdom), published on Mar. 24, 2007, which is incorporated by reference in its entirety, and disclosed in Kokoris, Mark (2025), “Sequencing by Expansion (SBX) – a novel, high-throughput single-molecule sequencing technology”, bioRxiv 2025.02.19.639056; doi: https://doi.org/10.1101/2025.02.19.639056, which is incorporated by reference in its entirety. Examples of MspA protein pores are disclosed in U.S. Patent 8,673,550 B2, entitled “MSP Nanopores and Related Methods,” assigned to University of Washington (Washington, United States) and to The UAB Research Foundation (Alabama, United States), issued on Mar. 18, 2013, which is incorporated by reference in its entirety.

Examples of membranes, such as amphiphilic polymer layers, solid-state membranes, and other suitable membranes are disclosed in GARNI et al., “Biopores/membrane proteins in synthetic polymer membranes,” Biochimica Et Biophysica Acta, Elsevier, Amsterdam, Nl, 1859(4): pp. 619-638 (2016) XP029914706 DOI: 10.1016/J.BBAMEM.2016.10.015, which is incorporated by reference in its entirety.

The membrane 130 separates the nanopore unit cell into a cis well 122 and a trans well 124. An analyte 160, such as a polynucleotide or a polypeptide, can translocate relative to biological nanopore 150, such as towards the trans well 124 and/or towards the cis well 122. The cis electrode and a trans electrode 122 may be used to apply a bias across the biological nanopore 150. The electrodes may be non-faradaic electrodes or faradaic electrodes. For example, in some embodiments, both the cis electrode and trans electrodes can be non-faradaic electrodes. In some embodiments, the cis electrode can be a non-faradaic electrode, and the trans electrodes can be faradaic electrodes. In some embodiments, the cis electrode can be a faradaic electrode, and the trans electrodes can be non-faradaic electrodes. A current, resistance, impedance, voltage or other electrical characteristic associated with the analyte in the biological nanopore 150 may be measured by the trans electrode 122 or other electrical component.

For example, when a bias is applied across the biological nanopore 150, a polynucleotide analyte or polypeptide analyte may translocate relative to the nanopore 150. When a nucleotide or a sequence combination of nucleotides of the polynucleotide analyte or a residue or a sequence combination of residues of the polypeptide analyte is in or near the nanopore 150, it may result in a unique blockade at the nanopore 150 and, therefore, a unique nanopore electrical characteristic depending on the identity of the nucleotide or the sequence combination of nucleotides of the polynucleotide analyte or on the identity of the residue or the sequence combination of residues of the polypeptide analyte. By measuring the electrical characteristic, the nucleotide or the sequence combination of nucleotides analyte or the residue or the sequence combinations of residue of the polypeptide analyte at or near the nanopore can be identified. In other words, the polynucleotide or polypeptide translocating relative to the nanopore may modulate the electrical properties of the nanopore such that the nucleobase sequence of the polynucleotide or amino acid sequence of the polypeptide can be identified. Furthermore, for the same polynucleotide or for the same polypeptide, the electrical characteristic may be different when the polynucleotide or polypeptide is translocating the nanopore in different directions.

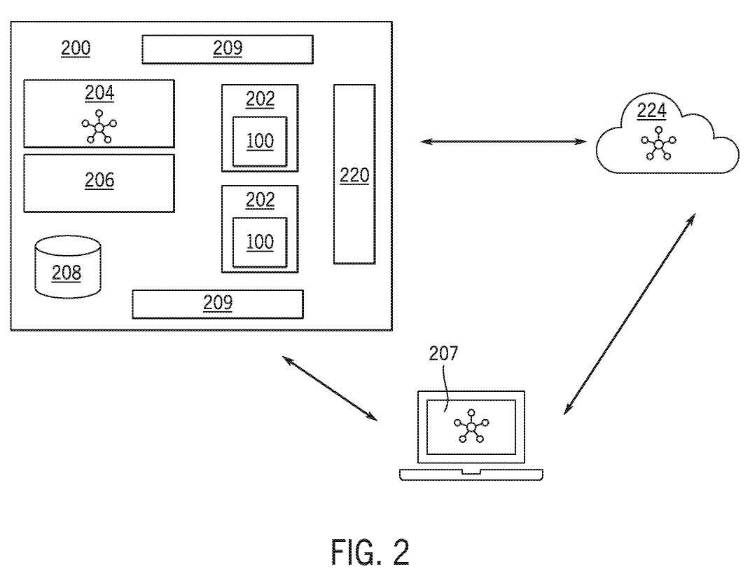

FIG. 2 is a schematic illustration of certain embodiments of a nanopore sequencing system 200. The nanopore sequencing system 200 includes one or more nanopore flow cell supports 202 to support one or more flow cells, such as the flow cells 100 as described in FIGS. 1A-C. The nanopore sequencing system 200 may further include an on-board controller 204. The controller 204 may be configured to control the sequencing operations in the array of nanopore unit cells. In certain embodiments, the nanopore sequencing system 200 may include a local interface 209 to couple to a local external controller 207, such as a computer or local server. In certain embodiments, the nanopore sequencing system 200 may include a cloud interface 220 to couple to a cloud controller 224. In certain embodiments, the nanopore sequencing system 200 may further include memory 206 and/or data storage 208.

One or more of the controllers 204, 207, 224 perform one or more elements of the method and process described herein. In some examples, the control functionalities of controllers 204, 207, 224 can be implemented or performed by a machine, such as a processor configured with specific instructions, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A processor can be a microprocessor, but in the alternative, the processor can be a controller, microcontroller, or state machine, combinations of the same, or the like. A processor or group of processors for performing the methods described herein may be of various types, including programmable devices (e.g., CPLDs and FPGAs) and non-programmable devices such as gate array ASICs or general-purpose microprocessors.

A processor can also be implemented as a combination of computing devices, e.g., a combination of a DSP and a microprocessor, a plurality of microprocessors, one or more microprocessors in conjunction with a DSP core, or any other such configuration. For example, systems described herein may be implemented using a discrete memory chip, a portion of memory in a microprocessor, flash, EPROM, or other types of memory. In some examples, a hardware platform for providing a computational environment may be used. The hardware platform may comprise a processor (e.g., CPU) and a memory such as random-access memory (RAM). In some embodiments, graphics processing units (GPUs) can be used. In some embodiments, hardware platforms for performing computational methods as described herein comprise one or more computer systems with one or more processors. In some embodiments, smaller computers are clustered together to yield a supercomputer network. The hardware platform may be specially constructed for the required purposes, or it may be a general-purpose computer (or a group of computers) selectively activated or reconfigured by a computer program and/or data structure stored in the computer. In some embodiments, a group of processors performs some or all of the described functionalities collaboratively (e.g., via a network or cloud computing) and/or in parallel.

Elements of the methods or processes described herein can be embodied in a software module executed by a processor. A software module can reside in RAM memory, flash memory, ROM memory, EPROM memory, EEPROM memory, registers, hard disk, a removable disk, a CD-ROM, or any other form of computer-readable storage medium. An exemplary storage medium can be coupled to the processor such that the processor can read information from, and write information to, the storage medium. In the alternative, the storage medium can be integral to the processor. The processor and the storage medium can reside in an ASIC. A software module can comprise computer-executable instructions which cause a hardware processor to execute the computer-executable instructions.

### Analytes

Sequencing of an analyte may involve nanopore sensing of (1) an elongated polymer; (2) a single-stranded portion of a polynucleotide; (3) a nucleic acid duplex of a portion of the polynucleotide; (4) a label or tag that can be tethered or untethered to the polynucleotide or polypeptide; (5) chemical modifications thereof, or (6) any combination thereof.

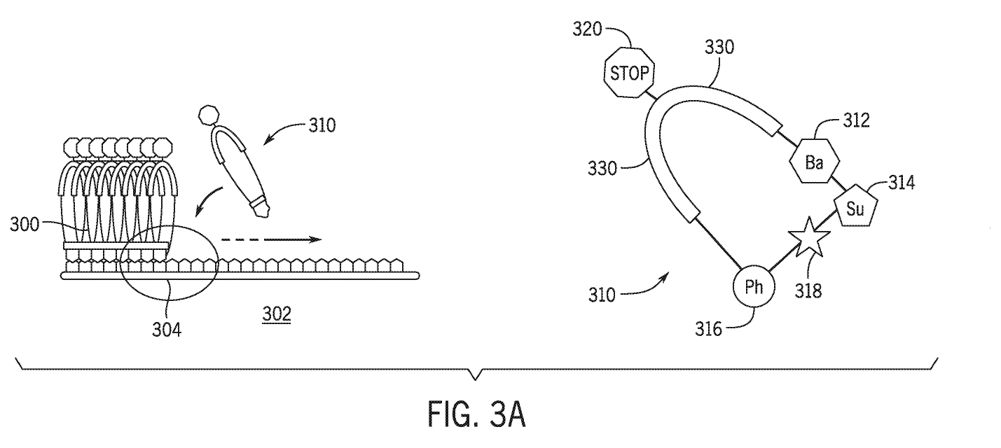

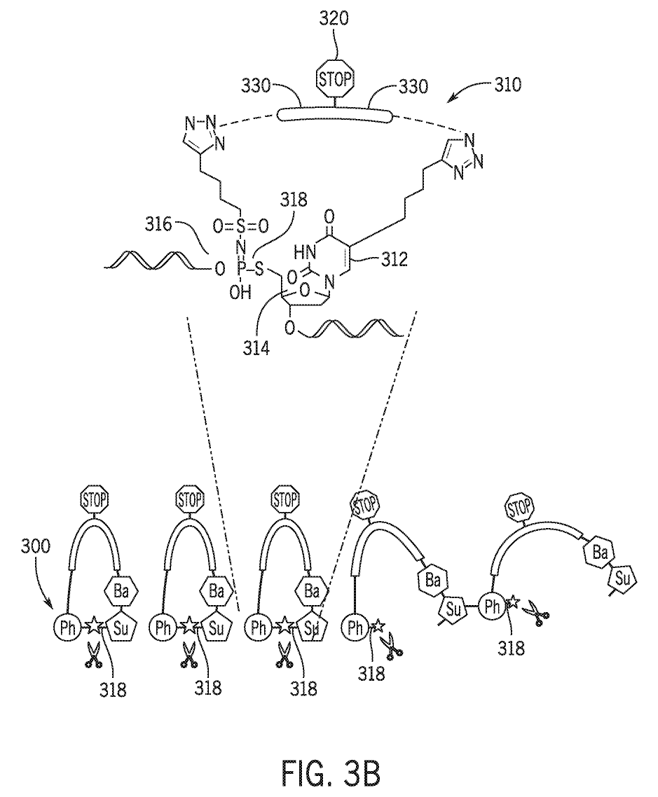

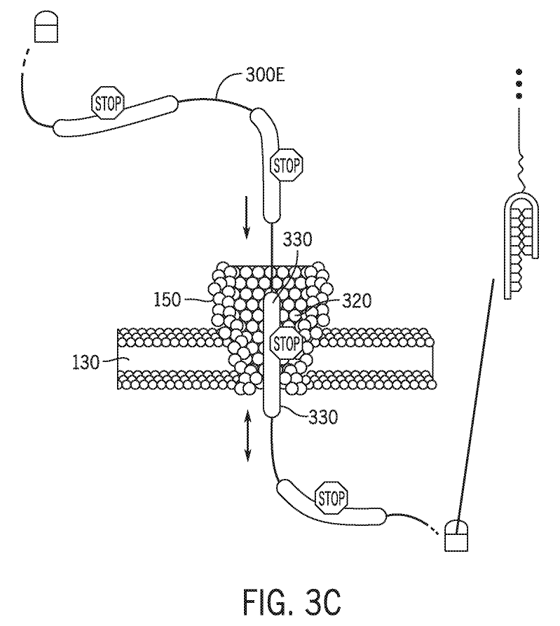

FIGS. 3A-C are schematic illustrations of certain embodiments of preparing and sequencing of an elongated polymer, such as an elongated polynucleotide. In FIG. 3A, a daughter strand 300 is synthesized using cyclic loop nucleotides (CLNs) 310 and the elongated daughter strand is sequenced instead of directly sequencing the template polynucleotide. Each CLNs is a modified nucleotide. As shown in FIG. 3A, each CLNs 310 comprises a base 312, a sugar 314, phosphates 316, and a cleavage site 318. A daughter strand 300 can be synthesized by a polymerase using CLNs 310 from a template polynucleotide 302 in which the CLNs are incorporated into a growing daughter strand 300.

Each CLNs 310 may further comprises one or more arresting constructs 320 that can arrest or slow translocation of the daughter strand 300, such as an elongated polynucleotide after cleavage of the CLNs at the cleavage sites 318, when encountering the nanopore. CLNs comprising arresting constructs 320 are described in PCT International Published Patent Application WO2024/228928 A1, entitled “Controlled Polynucleotide Translocation in Nanopore Sequencing”, filed by applicant Illumina, Inc. (San Diego, California, United States of America), published on Nov. 7, 2024 and in PCT International Published Patent Application WO2025/072861A1, entitled “Cleavable Cyclic Loop Nucleotides for Nanopore Sequencing”, filed by applicant Illumina, Inc. (San Diego, California, United States of America), published on Apr. 3, 2025, both of which are incorporated by reference in its entirety. Other embodiments of arresting constructs that can arrest or slow translocation the elongated polynucleotide are described in Kokoris, Mark (2025), “Sequencing by Expansion (SBX) – a novel, high-throughput single-molecule sequencing technology” and described in PCT International Published Patent Application WO2020/23656 A1, entitled “Translocation Control Elements, Reporter Codes, and Further Means for Translocation Control for Use in Nanopore Sequencing”, filed by Stratos Genomics, Inc. (Seattle, Washington, United States of America), published on Nov. 26, 2010, both of which are incorporated by reference in their entirety.

In some embodiments, each CLNs 310 may further comprise a set of one or more reporters 330 forming a unique barcoding/reporter region that is specific to the original bases (e.g., A, T, C, or G). Two reporters 330 are shown in FIG. 3A with an arresting construct 320 therebetween. When an CLN 310 comprises two reporters 330, the reporters may be the same or different. When the CLN 310 comprises three or more reporters 330, the reporters may be all the same, partially the same/different, or all different.

FIG. 3B is a schematic illustration of certain embodiments of the daughter analyte strand 300 of a target single stranded polynucleotide. After the daughter strand 300 is made or polymerized, the polynucleotide backbone is cleaved at cleavage sites 318, allowing the cyclic loop modification to open and resulting in elongation of the daughter strand polynucleotide 300 to form an elongated polymer. The cyclic loop modifications on the modified dNTPs become the linker constructs in the elongated polynucleotide 300E that create distance between adjacent nucleotides. Cleavage of the daughter strand at cleavage sites can be designed to occur at any part of the polynucleotide backbone as long as it occurs within the loop structure between the two positions where the linker construct is attached to the nucleotide structure and can be designed using any suitable cleavage chemistry. FIG. 3B shows one example of the cleavage site 318 between the phosphate groups 316 and the sugar 314. FIG. 3B shows one example of the cleavage chemistry of alpha-phosphate of imino-PS, which is disclosed in CLNs are disclosed in PCT International Patent Application Publication WO2023/215506 A1, entitled “Cleavable Cyclic Loop Nucleotides for Nanopore Sequencing”, filed by applicants Illumina, Inc. (San Diego, California, United States of America) and Illumina Singapore Pte. Ltd. (Singapore), published on November 9, 2023, which is incorporated by reference in its entirety. Other examples of cleavage chemistries are disclosed in PCT International Patent Application Publication WO 2008/157696 A2, entitled “High Throughput Nucleic Acid Sequencing by Expansion”, filed by applicant Stratos Genomics Inc. (Seattle, Washington, United States of America), published on Dec. 24, 2008, which is incorporated by reference in its entirety

FIG. 3C is a schematic illustration of certain embodiments of an elongated polymer, elongated daughter strand 300E of the target single stranded polynucleotide. Cleavage of the daughter strand along the backbone opens the loops and elongates the daughter strand leaving an elongated polymer. In embodiments where the linker construct contains an arresting construct 330 to interact with nanopore 150, the arresting construct may slow or halt the translocation of the elongated polymer and allow the nucleotides to be read by the nanopore one nucleotide at a time or one group of nucleotides at a time. In embodiments where the linker construct contains a reporter(s) 330, the elongated polymer exposes a series of reporters in which one or more reporters are used to identify a corresponding base. In embodiments where the linker construct contains an arresting construct 330 to interact with nanopore 150 and a reporter(s) 330, the arresting construct may slow or halt the translocation of the elongated polymer and allow the reporters to reside in and be read by the nanopore one reporter at a time or one group of reporters at a time.

As used herein, an “elongated polymer” is intended to mean any chain of elongated units in which the elongated polymer corresponds to a sequence of nucleotides in a polynucleotide, to sequence of a polypeptide, or to a sequence of nucleic acid-peptide conjugates. An XPANDOMER™ is a particular type of elongated polymer from Roche Sequencing, Inc. (Pleasanton, CA). XPANDOMER™ are described in “Kokoris, Mark (2025), Sequencing by Expansion (SBX) – a novel, high-throughput single-molecule sequencing technology.”

The daughter strand 300 is then “elongated” by cutting the cleavable sites. Consequently, when sequencing the daughter strand, the nanopore can “read” the barcoding/reporter region to identify the base that it is coding for. The linker and barcoding construct that is introduced in the daughter strand via polymerization is designed to occupy the readhead of the nanopore entirely, hence reducing the number of signals to just four, i.e., one per nucleobase. Thus, the disclosed technology allows barcode-based decoding of individual bases. In some embodiments, the cyclic loops contain non-barcoding linker constructs which allow the daughter strand to elongate after cutting the cleavable sites. The non-barcoding linker constructs may produce a distinguishable signal or a distinguishable signal break from signals of the nucleobases when passing through the nanopore, thereby isolating and/or enhance the recorded signals from the nucleobases.

### YAM Sequencing

In other embodiments, nanopore sequencings involves sensing a polynucleotide that includes a formed duplex. The duplex may be extended with a polymerase during sequencing. When a potential is applied across a nanopore, the duplex or extended duplex is held within the nanopore since the size of the constriction of the nanopore inhibits passage of the duplex completely through the nanopore. The electrical signal of the duplex held within the nanopore is used to identify the polynucleotide sequencing. Detecting a polynucleotide duplex is described in PCT International Published Patent Application WO2023/049682A1A1 by applicant Illumina, Inc. (San Diego, California, United States of America).

### ONT Long-Read Strand Sequencing

In other embodiments, nanopore sequencing is long-read strand sequencing. One example of long-read strand sequencing involves the use of a polynucleotide binding protein, such as a motor protein or a helicase to control the movement of the polynucleotide through the nanopore. The polynucleotide binding protein may be used to simultaneously separate the double stranded polynucleotide and control the rate of translocation of the resultant single strand through the nanopore. When a potential is applied across a nanopore, there is a change in the current flow when an analyte, such as a nucleotide, resides transiently in nanopore for a certain period of time. The single polynucleotide strand is passed through the pore and the identities of the nucleotides are derived. Nanopore sequencing instruments that employ long-read strand sequencing includes the MinION^{TM}, GridION^{TM}, and PromethION^{TM} from Oxford Nanopore Technologies (Oxford, United Kingdom) and the CycloneSEQ^{TM} from BGI Group (Shenzhen, China) and its subsidiaries.

### TERMS

As used herein, a “nucleotide” includes a nitrogen containing heterocyclic base, a sugar, and one or more phosphate groups. Nucleotides are monomeric units of a nucleic acid sequence. Examples of nucleotides include, for example, ribonucleotides or deoxyribonucleotides. In ribonucleotides (RNA), the sugar is a ribose, and in deoxyribonucleotides (DNA), the sugar is a deoxyribose, i.e., a sugar lacking a hydroxyl group that is present at the 2’ position in ribose. The nitrogen containing heterocyclic base can be a purine base or a pyrimidine base. Purine bases include adenine (A) and guanine (G), and modified derivatives or analogs thereof. Pyrimidine bases include cytosine (C), thymine (T), and uracil (U), and modified derivatives or analogs thereof. The C-l atom of deoxyribose is bonded to N-l of a pyrimidine or N-9 of a purine. The phosphate groups may be in the mono-phosphate, di-phosphate, tri-phosphate, or four or more phosphates. Nucleotides include deoxyribonucleotides, modified deoxyribonucleotides, ribonucleotides, modified ribonucleotides, peptide nucleotides, modified peptide nucleotides, modified phosphate sugar backbone nucleotides, and mixtures thereof. In some embodiments, the nucleotide may be an abasic nucleotide lacking a nucleobase.

As used herein, the term “nucleotide” also is intended to encompass any nucleotide analogue which is a type of nucleotide that includes a modified nucleobase, sugar, backbone, and/or phosphate moiety compared to naturally occurring nucleotides. Nucleotide analogues also may be referred to as “modified nucleic acids.” Example modified nucleobases include inosine, xanthine, hypoxanthine, isocytosine, isoguanine, 2-aminopurine, 5-methylcytosine, 5-hydroxymethyl cytosine, 2-aminoadenine, 6-methyl adenine, 6-methyl guanine, 2-propyl guanine, 2-propyl adenine, 2-thiouracil, 2-thiothymine, 2-thiocytosine, 15-halouracil, 15-halocytosine, 5-propynyl uracil, 5-propynyl cytosine, 6-azo uracil, 6-azo cytosine, 6-azo thymine, 5-uracil, 4-thiouracil, 8-halo adenine or guanine, 8-amino adenine or guanine, 8-thiol adenine or guanine, 8-thioalkyl adenine or guanine, 8-hydroxyl adenine or guanine, 5-halo substituted uracil or cytosine, 7-methylguanine, 7-methyladenine, 8-azaguanine, 8-azaadenine, 7-deazaguanine, 7-deazaadenine, 3-deazaguanine, 3-deazaadenine or the like. As is known in the art, certain nucleotide analogues cannot become incorporated into a polynucleotide, for example, nucleotide analogues such as adenosine 5ʹ-phosphosulfate. Nucleotide analogues also include locked nucleic acids (LNA), peptide nucleic acids (PNA), and 5-hydroxylbutynl-2'-deoxyuridine (“super T”).

As used herein, the term “polynucleotide” refers to a molecule that includes a sequence of nucleotides that are bonded to one another. A polynucleotide is one nonlimiting example of a polymer. Examples of polynucleotides include deoxyribonucleic acid (DNA), ribonucleic acid (RNA), and analogues thereof such as locked nucleic acids (LNA) and peptide nucleic acids (PNA). A polynucleotide may be a single stranded sequence of nucleotides, such as RNA or single stranded DNA, a double stranded sequence of nucleotides, such as double stranded DNA, or may include a mixture of a single stranded and double stranded sequences of nucleotides. Double stranded DNA (dsDNA) includes genomic DNA, and PCR and amplification products. Single stranded DNA (ssDNA) can be converted to dsDNA and vice-versa. Polynucleotides may include non-naturally occurring DNA, such as enantiomeric DNA, LNA, or PNA. The precise sequence of nucleotides in a polynucleotide may be known or unknown. The following are examples of polynucleotides: a gene or gene fragment (for example, a probe, primer, expressed sequence tag (EST) or serial analysis of gene expression (SAGE) tag), genomic DNA, genomic DNA fragment, exon, intron, messenger RNA (mRNA), transfer RNA, ribosomal RNA, ribozyme, cDNA, recombinant polynucleotide, synthetic polynucleotide, branched polynucleotide, plasmid, vector, isolated DNA of any sequence, isolated RNA of any sequence, nucleic acid probe, primer or amplified copy of any of the foregoing.

As used herein, a “polymerase” is intended to mean an enzyme having an active site that assembles polynucleotides by polymerizing nucleotides into polynucleotides. A polymerase can bind a primer and a single stranded target polynucleotide and can sequentially add nucleotides to the growing primer to form a “complementary copy” polynucleotide having a sequence that is complementary to that of the target polynucleotide. DNA polymerases may bind to the target polynucleotide and then move down the target polynucleotide sequentially adding nucleotides to the free hydroxyl group at the 3ʹ end of a growing polynucleotide strand. DNA polymerases may synthesize complementary DNA molecules from DNA templates. RNA polymerases may synthesize RNA molecules from DNA templates (transcription). Other RNA polymerases, such as reverse transcriptases, may synthesize cDNA molecules from RNA templates. Still other RNA polymerases may synthesize RNA molecules from RNA templates, such as RdRP. Polymerases may use a short RNA or DNA strand (primer), to begin strand growth. Some polymerases may displace the strand upstream of the site where they are adding bases to a chain. Such polymerases may be said to be strand displacing, meaning they have an activity that removes a complementary strand from a template strand being read by the polymerase.

Example DNA polymerases include members of the Y-family, such as Dpo4 polymerase (Sulfolobus solfataricus); B-family polymerases, such as 9° N; and A-family polymerases, such as Bsu. Additional examples include Bst DNA polymerase, Phi29 DNA polymerase, DNA polymerase I (E. coli), DNA polymerase I (Large Fragment), Klenow fragment (including 3′–5′ exo- variants), T4 DNA polymerase, T7 DNA polymerase, Deep VentR™ (exo-) DNA polymerase, Deep VentR™ DNA polymerase, DyNAzyme™ EXT DNA polymerase, DyNAzyme™ II Hot Start DNA polymerase, Phusion™ High-Fidelity DNA polymerase, Therminator™ DNA polymerase, Therminator™ II DNA polymerase, VentR® DNA polymerase, VentR® (exo-) DNA polymerase, RepliPHI™ Phi29 DNA polymerase, rBst DNA polymerase (including Large Fragment), IsoTherm™ DNA polymerase, MasterAmp™ AmpliTherm™ DNA polymerase, Taq DNA polymerase, Tth DNA polymerase, Tfl DNA polymerase, Tgo DNA polymerase, SP6 DNA polymerase, Tbr DNA polymerase, DNA polymerase Beta, ThermoPhi DNA polymerase, and Isopol™ SD+ polymerase. Some polymerases have an activity that degrades the strand behind them (3ʹ exonuclease activity). Some useful polymerases have been modified, either by mutation or otherwise, to reduce or eliminate 3ʹ and/or 5ʹ exonuclease activity.

As used herein, the term “primer” is defined as a polynucleotide to which nucleotides may be added via a free 3ʹ OH group. A primer may include a 3ʹ block inhibiting polymerization until the block is removed. A primer may include a modification at the 5ʹ terminus to allow a coupling reaction or to couple the primer to another moiety. A primer may include one or more moieties, such as 8-oxo-G, which may be cleaved under suitable conditions, such as UV light, chemistry, enzyme, or the like. The primer length may be any suitable number of bases long and may include any suitable combination of natural and non-natural nucleotides. A target polynucleotide may include an “amplification adapter” or, more simply, an “adapter,” that hybridizes to (has a sequence that is complementary to) a primer, and may be amplified so as to generate a complementary copy polynucleotide by adding nucleotides to the free 3ʹ OH group of the primer.

As used herein, the term “double-stranded,” when used in reference to a polynucleotide or portion thereof, is intended to mean that all or substantially all of the nucleotides in the polynucleotide or section thereof are hydrogen bonded to respective nucleotides in a complementary polynucleotide. A double-stranded polynucleotide also may be referred to as a “duplex.”

As used herein, the term “single-stranded,” when used in reference to a polynucleotide, means that essentially none of the nucleotides in the polynucleotide or section thereof are hydrogen bonded to a respective nucleotide in a complementary polynucleotide.

As used herein, the term “target polynucleotide” is intended to mean a polynucleotide that is the object of an analysis or action and may also be referred to using terms such as “library polynucleotide,” “template polynucleotide,” or “library template.” The analysis or action includes subjecting the polynucleotide to amplification, sequencing and/or other procedure. A target polynucleotide may include nucleotide sequences additional to a target sequence to be analyzed. For example, a target polynucleotide may include one or more adapters, including an amplification adapter that functions as a primer binding site, that flank(s) a target polynucleotide sequence that is to be analyzed. In particular examples, target polynucleotides may have different sequences than one another but may have first and second adapters that are the same as one another. The two adapters that may flank a particular target polynucleotide sequence may have the same sequence as one another, or complementary sequences to one another, or the two adapters may have different sequences. Thus, species in a plurality of target polynucleotides may include regions of known sequence that flank regions of unknown sequence that are to be evaluated by, for example, nanopore sequencing. In some examples, target polynucleotides carry an amplification adapter at a single end, and such adapter may be located at either the 3ʹ end or the 5ʹ end the target polynucleotide. Target polynucleotides may be used without any adapter, in which case a primer binding sequence may come directly from a sequence found in the target polynucleotide.

The terms “polynucleotide” and “oligonucleotide” are used interchangeably herein. The different terms are not intended to denote any particular difference in size, sequence, or other property unless specifically indicated otherwise. For clarity of description, the terms may be used to distinguish one species of polynucleotide from another when describing a particular method or composition that includes several polynucleotide species.

A “polynucleotide nanopore” is intended to mean a nanopore that is made from one or more nucleic acid polymers. A polynucleotide nanopore can include, for example, a polynucleotide origami.

### Equivalent Circuit and Stimulus of a Nanopore

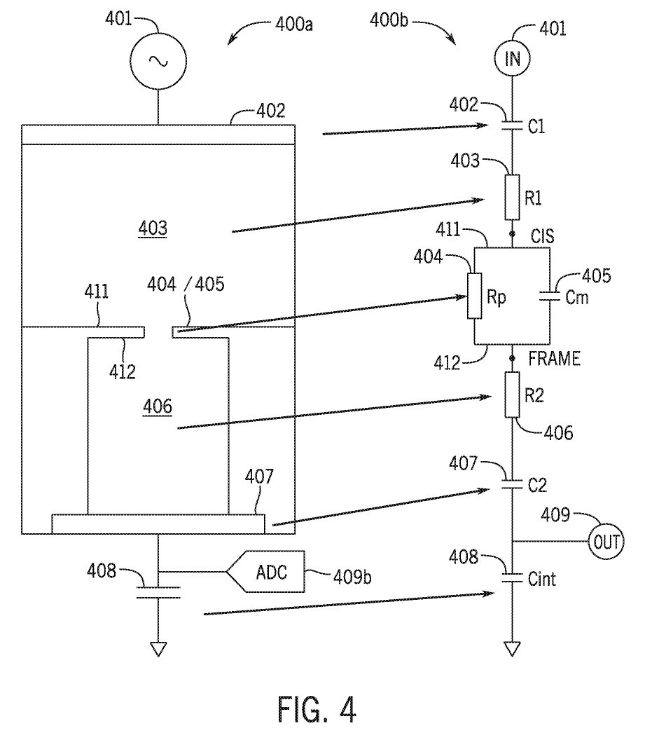

FIG. 4 depicts a physical diagram of a nanopore 400a and a corresponding electrical circuit model 400b. Beginning at the top of FIG. 4, the nanopore and electrical circuit include an “input” node or top electrode 401. Unless expressly noted or clearly required by context, directional descriptions herein, such as “top,” “bottom,” “above,” “below,” etc. are merely relative to the depictions in the figures and a physical implementation of the discussed concepts could be in any suitable orientation. As described in greater detail herein, this input node can be but need not necessarily be the point at which a stimulus signal is input into the nanopore. In some embodiments it can be a terminal that can be kept at a reference voltage, i.e., an AC ground, but which can be at any suitable DC potential, including but not limited to DC ground. This input node 401 can be connected to a first double layer capacitance 402. This double layer capacitance 402 (and the other double layer capacitance 407, discussed below) are present at any electrode-electrolyte interface. These double-layer capacitances may be effectively short-circuited when Faradaic electrodes are used.

Double layer capacitance 402 couples to a cis or common tank or well 403, which can be filled with a suitable working fluid, as described above. This cis well can be modeled as an equivalent resistance R1/403. Below this cis well 403 is the nanopore 404/405, which can be constructed as described above and can be electrically modeled as a parallel pore resistance Rp/404 and membrane capacitance Cm/405. The pore resistance Rp/404 varies with the particular analyte (e.g., a nucleotide/reporter construct) that is translocated with respect to a particular nanopore. As described elsewhere herein, a stimulus signal applied to the nanopore can be used to characterize this resistance using the circuit response to the stimulus signal, thereby allowing identification of the nucleotide (analyte) that is presently translocated with respect to the nanopore. (For purposes of the following description, identification of a “nucleotide” and/or “reporter” should be understood as encompassing identification of any analyte corresponding to a particular sequencing biochemistry.) With further reference to FIG. 4, the upper (in FIG. 4) side of the nanopore can be called a cis electrode 411, and the lower (in FIG. 4) side of the nanopore can be called the trans electrode 412.

Proceeding downward (in FIG. 4), the trans electrode sits above a trans tank or well 406, also filled with a suitable working fluid. This trans well can be modeled as a resistance R2/406. Then, below (in FIG. 4) this, is the second lipid bilayer capacitance C2/407. A bottom electrode or “output” node 409 can be coupled to this bilayer capacitance. Although described as an “output,” in some embodiments a stimulus signal can be applied to terminal 409 as well as having a response to the stimulus signal (e.g., an output signal, such as a voltage, current, resistance, etc.) being read, e.g., by an analog to digital converter “ADC” 409b. Also present in at least some embodiments can be an integration capacitor Cint/408, which can be used to store the response to the stimulus signal as briefly described below. Finally, the output side of the integration capacitor Cint/408 can be coupled to a reference node that can again be grounded or at any suitable reference voltage. For purposes of FIG. 4 and all circuits depicted or described in the present application, all capacitors are depicted as “ideal” capacitors, without any faradaic or leakage currents or other non-idealities. In a real system, such non-idealities may be present, but their presence will not materially affect the embodiments and operating principles described herein.

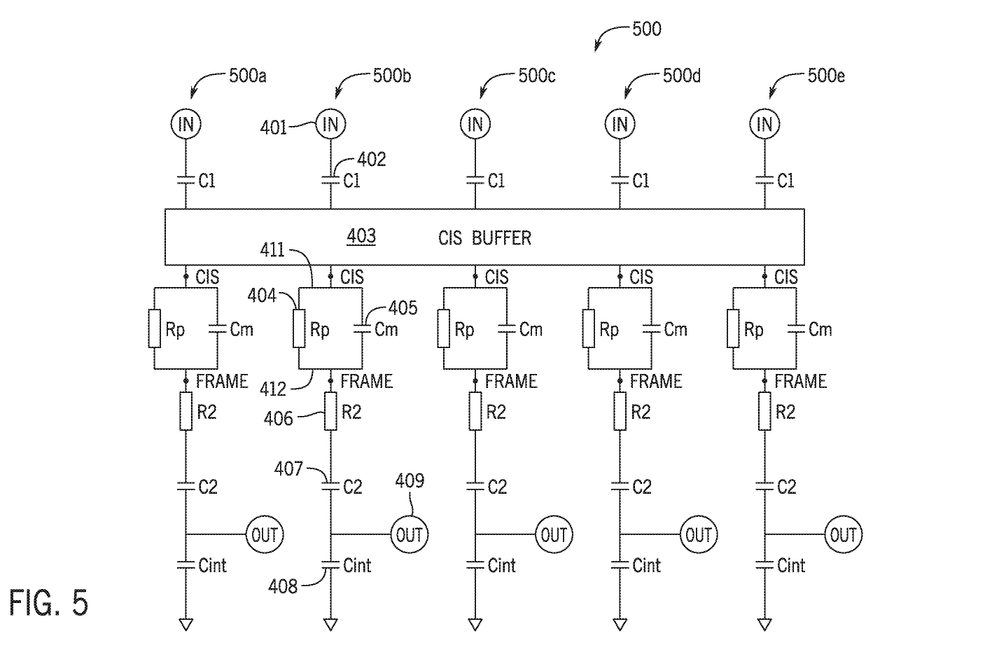

FIG. 5 depicts a simplified schematic 500 of a plurality of nanopores 500a–500e, representing a plurality of nanopores arranged, for example, on an integrated circuit as described above. Reference numbers corresponding to FIG. 4 are provided for nanopore 500b, and it should be understood that the same reference numbers apply equally to nanopores 500a, and 500c–500e but have been omitted to avoid cluttering FIG. 5. FIG. 5 further depicts that cis buffer 403 located in the cis well or tank described above with reference to FIG. 4 is common to all or at least a plurality of the nanopores. As a result, the respective nanopores are not electrically isolated from one another, and stimulus signals applied to particular nanopores may affect other nanopores as well. This phenomenon may be thought of as pixel-to-pixel crosstalk in a nanopore integrated circuit constructed as described above, and various operational techniques can be employed to account for this crosstalk.

When operating a nanopore as described and modeled above, a stimulus signal may be applied to the top electrode, and the resulting circuit behavior measured at the bottom electrode to identify a nucleotide/reporter translocated with respect to the nanopore. Alternatively, the top electrode can be held at a reference potential, and the stimulus signal may be applied to the bottom electrode during a first portion of a cycle, and the response (e.g., output signal) measured at the same bottom electrode during a second portion of the cycle. Thus, the stimulus and read operations are interleaved. When applied to an array of nanopores, such as a nanopore array constructed on an integrated circuit as described above, each measurement cycle may be thought of as a “frame.” During each frame, the stimulus may be applied to all nanopores simultaneously, e.g., “global” stimulus or may be applied to some, but not all nanopores of nanopores in a rotating cycle, e.g., “rolling” stimulus. In either case (global or rolling stimulus), different stimulus signals can be applied to different nanopores or groups of nanopores at the same time. As one non-limiting example, in a global stimulus context, positive stimulus voltages could be applied to one subset of the nanopores while negative stimulus voltages are applied to another subset of the nanopores, with all nanopores or groups of nanopores still being stimulated simultaneously. In another non-limiting example, in a rolling stimulus context, positive stimulus voltages could be applied to one subset o the nanopores while negative stimulus voltages are applied to another subset of nanopores, with some nanopores not being stimulated during this period. Similarly, the measurement operation may be performed on all nanopores simultaneously, e.g., global measurement or global shutter, or the measurement may be performed on a subset of nanopores in a rotating cycle, e.g., a rolling measurement, or rolling shutter.

Stimulus signals applied to the nanopores can include stimulus pulses (such as the examples described herein) for sensing and readout of analytes translocated with respect to the nanopores. Additionally, other electrical signals or waveforms may be applied to one or more cis/common electrodes and/or trans electrodes for performing additional nanopore unit cell operations. Such operations may include, but are not limited to, membrane formation, biological pore insertion, analyte capture, analyte translocation, analyte ejection, membrane destruction, membrane refresh, or other nanopore unit cell initialization, maintenance, or reset operations. These additional signals waveforms may comprise voltage or current signals or waveforms of any suitable magnitude, polarity, duration, shape, or timing, and may be applied globally to all nanopores or selectively to individual nanopores or groups of nanopores, independently of or in coordination with the stimulus and readout operations described herein.

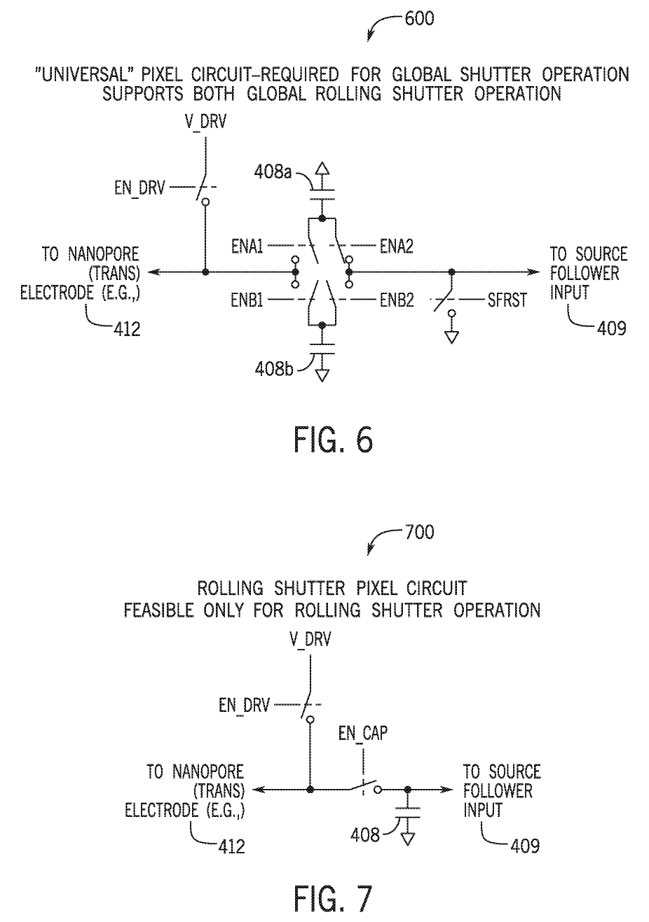

The stimulus of each nanopore produces an circuit response in the form of an output signal that can be measured to characterize the analyte (e.g., nucleotide/reporter) that is presently translocated with respect to the nanopore. Circuit responses may be transient responses, and thus the amount of time that has passed relative to the stimulus can affect the voltage, current, or other electrical parameters measured. Additionally, the capture and measurement of the response require a finite time to accomplish. As a result, to achieve a desired level of measurement accuracy when implementing a global shutter, a larger number and/or more complex measurement circuitry may be required. Conversely, a rolling shutter implementation can simplify and/or reduce the circuitry required. In some applications, a nanopore integrated circuit can be constructed with circuitry allowing for either global shutter or rolling shutter operation, as described in greater detail below with reference to FIGS. 6 and 7. In either case, for at least some applications, it may be advantageous to coordinate the measurement or shutter operation (whether global or rolling) with the timing of the analog to digital converter(s) (ADC(s)) that perform the measurement to achieve a desired level of accuracy.

FIG. 6 depicts a schematic 600 of a universal nanopore pixel circuit that can be used for either global shutter operation or rolling shutter operation. The leftmost terminal depicted in FIG. 6 can couple the pixel circuit to the trans electrode of a corresponding nanopore (e.g., via the bottom electrode and double layer capacitance). Also coupled to this node can be a drive switch that selectively couples a stimulus voltage V_DRV to the nanopore trans electrode responsive to an enable signal EN_DRV that selectively closes (and opens) the switch. In other embodiments, a stimulus signal could be applied to the common/cis electrode or terminal. The pixel circuit of FIG. 6 includes two integrating capacitors 408a/408b. Each integrating capacitor can be selectively coupled to either the nanopore trans electrode or the source follower input (e.g., output node ADC 409) by corresponding switches. For example, switch ENA1 can selectively couple integrating capacitor 408a to the nanopore trans electrode, and switch ENA2 can selectively couple integrating capacitor 408a to the output 409. Similarly, switch ENB1 can selectively couple integrating capacitor 408b to the nanopore trans electrode, and switch ENB2 can selectively couple integrating capacitor 408b to the output 409. An additional reset switch SFRST can be provided to selectively couple the output node 409 to ground as a reset.

To summarize, the universal nanopore pixel circuit can include a first switching device EN_DRV selectively operable to couple the input stimulus pulses to the trans electrode of the nanopore. The universal nanopore pixel circuit can further include a first integration capacitor 408a selectively couplable to the trans electrode 412 of the nanopore or the analog to digital converter (via source follower 409) by a first pair of complementarily operated switching devices ENA1/ENA2. By complementarily operated, it is meant that when ENA1 is closed to couple integrating capacitor 408a to the nanopore trans electrode 412, ENA2 is opened to decouple integrating capacitor 408a from the source follower and ADC, and vice-versa. The universal nanopore pixel circuit can further include a second integration capacitor 408b selectively couplable to the trans electrode 412 of the nanopore or the analog to digital converter (via source follower 409) by a second pair of complementarily operated switching devices ENB1/ENB2. By complementarily operated, it is meant that when ENB1 is closed to couple integrating capacitor 408b to the nanopore trans electrode 412, ENB2 is opened to decouple integrating capacitor 408b from the source follower and ADC, and vice-versa. Additionally, the respective complementarily operated switch pairs can be complementarily operated with respect to each other, meaning that when integrating capacitor 408a is coupled to the nanopore, integrating capacitor 408b can be coupled to the ADC via the source follower 409, and vice-versa.

In operation, the respective first and second integrating capacitors 408a/408b can be used to capture the response to stimulus signals correspond to alternating image frames. For example, the first integrating capacitor 408a can be used to capture the response to a first stimulus signal in a first image frame, while the second integrating capacitor 408b is coupled to the ADC, allowing the result of a prior image frame to be read. Then, in a subsequent frame, second integrating capacitor 408b can be used to capture the response to a second stimulus signal in a second image frame, while the first integrating capacitor 408a is coupled to the ADC, allowing the result of the first image frame to be read, and so on. In at least some embodiments, it may be desirable to control or regulate the timing of the integrating capacitors relative to the ADC readout schedule to improve measurement accuracy. This timing may vary depending on the number of ADCs employed, the number of nanopores and/or nanopore groups being stimulated simultaneously, etc.

FIG. 7 depicts a schematic 700 of a rolling shutter pixel circuit for rolling shutter operation. As with the universal pixel circuit described above with reference to FIG. 6, the leftmost terminal depicted in FIG. 7 can couple the pixel circuit to the trans electrode of a corresponding nanopore (e.g., via the bottom electrode and double layer capacitance). Also coupled to this node is a drive switch that selectively couples a stimulus voltage V_DRV to the nanopore trans electrode responsive to an enable signal EN_DRV that selectively closes (and opens) the switch. The rolling shutter pixel circuit of FIG. 7 includes a single integrating capacitor 408 that can be selectively coupled to the nanopore trans electrode by switch EN_CAP and is otherwise continuously coupled to the source follower input (e.g., output node ADC 409). In some embodiments, an optional reset switch and/or an optional switch for decoupling the integrating capacitor from the ADC could be provided. Additionally, the universal pixel circuit of FIG. 6 can be configured to become the rolling pixel circuit of FIG. 7, e.g., by opening switches ENA1 and ENA2 to isolate extra integration capacitor 408a and closing switch ENB2 to continuously couple integrating capacitor 408b to the ADC. Switch ENB1 can thus become the EN_CAP switch for selectively coupling the integrating capacitor to the nanopore trans electrode. As above with respect to FIG. 6, in at least some embodiments, it may be desirable to control or regulate the timing of the integrating capacitors relative to the ADC readout schedule to improve measurement accuracy. This timing may vary depending on the number of ADCs employed, the number of nanopores and/or nanopore groups being stimulated simultaneously, etc.

While it may be advantageous for development, testing, or other purposes to configure a universal pixel circuit to operate as a rolling shutter pixel circuit, in applications for which only rolling shutter operation is required, the simplified circuit of FIG. 7 can provide for a simpler, more compact, and less costly configuration as the unneeded integrating capacitor and switches can be omitted. This means a nanopore integrated circuit designed for rolling stimulus and/or rolling shutter operation (e.g., in which the stimulus signals to and/or the measurement of output signals from respective nanopores are staged as described herein) can contain more nanopores in a given area than an integrated circuit that supports global stimulus/shutter operation.

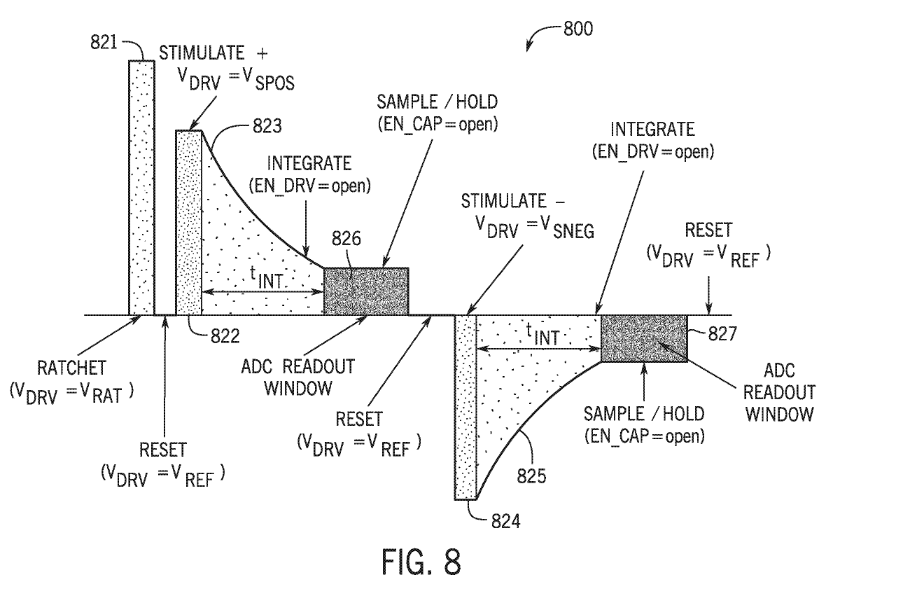

FIG. 8 depicts an example pixel stimulus for a nanopore, more specifically a differential voltage across a nanopore such as may be caused by trans-side stimulus of the nanopore. An initial ratchet pulse 821 can be used to translocate a new nucleotide/reporter with respect to the nanopore. A ratchet pulse refers to a controlled electrical voltage applied across a nanopore to advance a nucleic acid molecule, or an associated reporter construct, through the nanopore in a stepwise and repeatable manner. Each ratchet pulse applies a defined voltage waveform—typically of a specified polarity, amplitude, and duration—that transiently overcomes molecular or mechanical constraints (such as arresting constructs, duplex features, or enzyme-mediated interactions) to move the molecule to a next discrete position within or relative to the nanopore. The application of successive ratchet pulses allows the molecule to be incrementally advanced, such that each advancement produces a corresponding electrical signal characteristic of a specific nucleotide, reporter, or molecular feature. In this manner, ratchet pulses enable controlled translocation and sequencing by ensuring that molecular motion through the nanopore is synchronized with signal acquisition rather than translocation occurring continuously or stochastically. In other embodiments of sequencing biochemistry in which a nucleic acid molecule translocates continuously or stochastically, an example pixel stimulus for a nanopore will be similar to FIG. 8 but may omit the ratchet pulse 821.

In the example of FIG. 8, following the ratchet pulse 821 and also a reset interval, a stimulus pulse 822 can be applied, followed by an integration interval 823 during which the charge decays in the nanopore circuit, with the decay rate determined by the nanopore resistance (Rp), as all other circuit capacitances remain substantially the same without regard to the particular nucleotide/reporter translocated with respect to the nanopore. After the integration interval, the remaining voltage 826 can be measured during the ADC readout window.

In the illustrated example, the positive stimulus pulse 822 and associated integration and readout intervals can be followed by another reset interval and then a negative stimulus pulse 824. As described in greater detail below, applying stimulus pulses of alternating polarity can be used to offset drift and hysteresis effects associated with the nanopores. Following the negative stimulus pulse 824, an integration interval 825 can allow the voltage in the nanopore circuit to decay (as with the positive pulse), which can in turn be followed by a second ADC readout window 827 when the remaining voltage can be measured.

In the illustrated example of alternating positive and negative stimulus pulses, the positive measured voltage during the first ADC readout window 826 and the negative measured voltage during the second ADC readout window 827 can be used to determine the pore resistance Rp and thus the associated nucleotide/reporter that is presently translocated with respect to the nanopore. This can be based on various mathematical operations between the two measured voltages, such as a difference, a sum of absolute values, an average of absolute values, a sum of squares, etc. Additionally, although sequentially alternating positive and negative stimulus pulses are shown, the stimulus could include a sequence of multiple positive pulses (e.g., 3, 4, 5, 6, 7 or other number of positive stimulus pulses) followed by a sequence of multiple negative stimulus pulses (e.g., 3, 4, 5, 6, 7 or other number of negative stimulus pulses). In some embodiments, the number of sequential positive stimulus pulses can be equal to the number of sequential negative stimulus pulses.

### Rolling Stimulus, Global Shutter, and Rolling Shutter Operation

Whatever form the stimulus pulses applied to the nanopores takes, it may be desirable to stagger the stimulus to respective nanopores or groups of nanopores such that some nanopores or nanopore groups are being stimulated while others are not, described herein as “rolling stimulus.” This can achieve various operational advantages. For example, by stimulating certain nanopores or groups of nanopores together (while others are not stimulated) the crosstalk effects referenced above with respect to FIG. 5 can be mitigated. Likewise, by stimulating some nanopores or nanopore groups at different times, certain nanopores or nanopore groups can be read while others are being stimulated and vice-versa, and the number of circuit devices necessary for nanopore stimulus and readout can be reduced (because certain stimulation circuitry can be connected to nanopores being stimulated in each step, and certain readout circuitry can be connected to other nanopores being read out during the same interval).

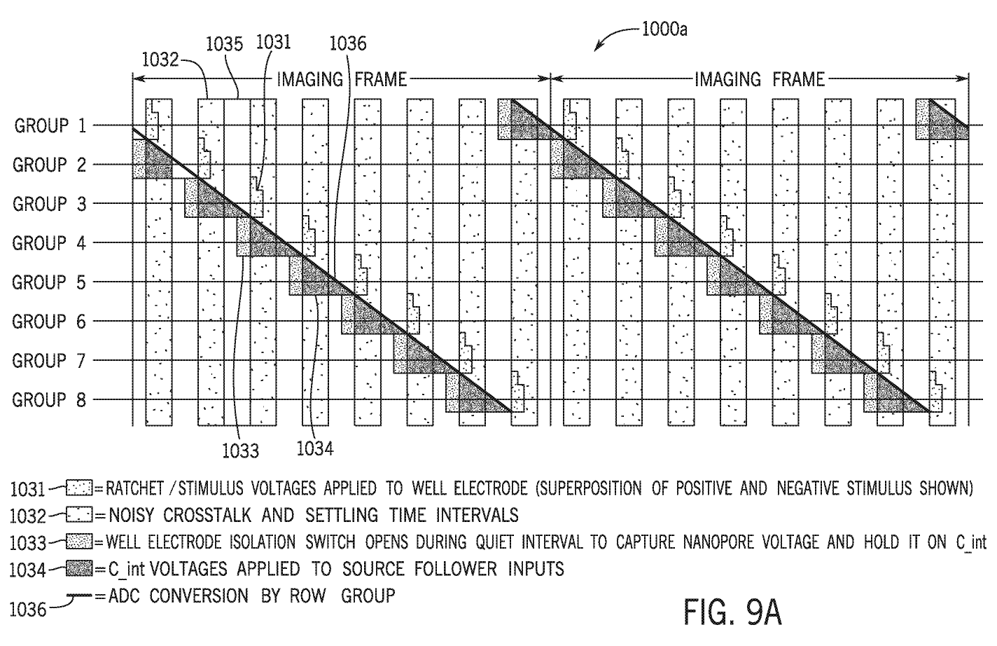

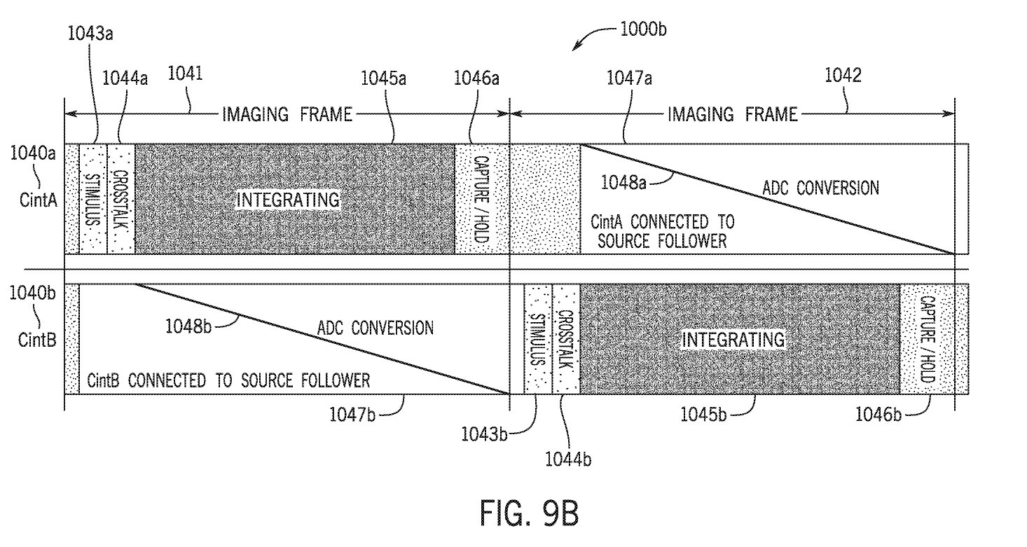

FIG. 9A depicts a rolling stimulus and rolling shutter operation 1000a for groups of nanopore pixels. More specifically, FIG. 9A depicts how crosstalk noise (corresponding to shaded regions 1032) is expected during and immediately after pulse injection for each pixel group. The unshaded regions 1035 represent the time window where noise has subsided, and voltages can be sampled for readout by the ADC. The diagonal line 1036 shows ADC readout timing, and the shaded stairstep regions 1031 show the rolling stimulus and rolling shutter timing that tracks the ADC readout.

Each pixel group is depicted in FIG. 9A by a horizontal line labeled Group 1 through Group 8. The horizontal axis of this plot corresponds to time, with two imaging frames (rolling shutter cycles) depicted. In the example of FIG. 9A, the rolling shutter is divided into eight sub-frames. As described above, when implementing a rolling shutter, only one of the respective integrating capacitors of a universal nanopore/pixel need be used. Returning to FIG. 9A, stimulus signals 1031 are depicted as the stairstep regions. For a first rolling shutter cycle (left-side imaging frame in FIG. 9A), the stimulus signal can include a ratchet pulse and one or more positive and/or negative stimulus pulses as described above, repeated with timings corresponding to the eight groups to apply the ratchet pulse and stimulus pulse(s) to each respective pixel group as a rolling stimulus. For a second rolling shutter cycle (right-side imaging frame in FIG. 9A), the stimulus signals can again be applied to respective pixel groups with timings staggered to apply the stimulus pulses to each respective pixel group as a rolling stimulus. Each pixel group is in turn stimulated by ratchet and stimulus pulses 1031, which occurs during a corresponding noisy crosstalk and settling time interval 1032, which can extend beyond the time that the ratchet and stimulus pulses are applied. Thereafter, in interval 1033, a well electrode isolation switch can open during quiet interval 1035 to capture the corresponding nanopore voltage and hold it (in the integrating capacitor) for readout by the analog to digital converter during interval 1034.

FIG. 9B depicts an alternative global shutter operation 1000b for nanopore pixels. The global shutter stimulus and sampling operation can be implemented with the universal pixel circuit described above with respect to FIG. 6. Two imaging frames 1041 and 1042 are depicted. Additionally, the use of the two separate integration capacitors 1040a/1040b (corresponding to capacitors 408a/408b) of a universal pixel circuit as described above with respect to FIG. 6) for each nanopore pixel are depicted in FIG. 9B. During a first imaging frame 1041, the first integrating capacitor 1040a can be coupled to the nanopore/pixel. A stimulus pulse 1043a can be applied, followed by a crosstalk damping interval 1044a allowing the crosstalk to dissipate or settle. Then, an integrating period 1045a can take place, which allows the response of the nanopore circuit to decay in response to the stimulus pulse until a capture/hold interval 1046a, during which the integration capacitor holds the value for readout by the ADC. Thereafter, during the second imaging frame 1042, the capacitor can be connected to the ADC (e.g., a source follower circuit of the integrated circuit) allowing for an ADC readout interval 1047a during which each pixel is read over a finite time period represented by ADC conversion line 1048a.

Operation of the second integration capacitor is complementary to that of the first. That is, when the first integrating capacitor 1040a is coupled to the nanopore/pixel (e.g., during first imaging frame 1041), the second integrating capacitor 1040b can be coupled to the ADC (e.g., via the source follower) to allow values corresponding to a previous image frame to be read out during ADC readout interval 1047b, with each pixel being read at a time depicted by line 1048b. Likewise, when the first integrating capacitor 1040a is coupled to the ADC (e.g., during imaging frame 1042), the second integrating capacitor can be coupled to the nanopore/pixel. A stimulus pulse 1043b can be applied, followed by a crosstalk damping interval 1044b allowing the crosstalk to dissipate or settle. Then, an integrating period 1045b can take place, which allows the response of the nanopore circuit to decay in response to the stimulus pulse until a capture/hold interval 1046b, during which the integration capacitor holds the value for readout by the ADC.

### Nanopore Time Constants, Hysteresis/Memory Effects and Compensation

As described above, each nanopore is a circuit including a pore resistance (Rp) a membrane capacitance (Cm) and other resistances (e.g., resistances associated with the cis and trans wells) and capacitances (e.g., double layer capacitances for non-Faradaic nanopores). Each of these components of the nanopore circuit affect time constants of the nanopore circuit that can lead to hysteresis or memory effects, in which the present value of a voltage or current in the nanopore is affected by prior stimulus or conditions. For instance, a short time constant associated with the pore resistance, membrane capacitance, and integration capacitor might be on the order of 50 to 500 microseconds. This time constant may dominate relaxation or decay of a sampled voltage during the ADC readout phase described above. A longer time constant associated with the double layer capacitances may be on the order of 10 to 50 milliseconds and can dominate during translocation (e.g., ratchet pulses), stimulation pulses, and reset periods. In some cases, it may be desirable to compensate for these effects, examples of which are described below.

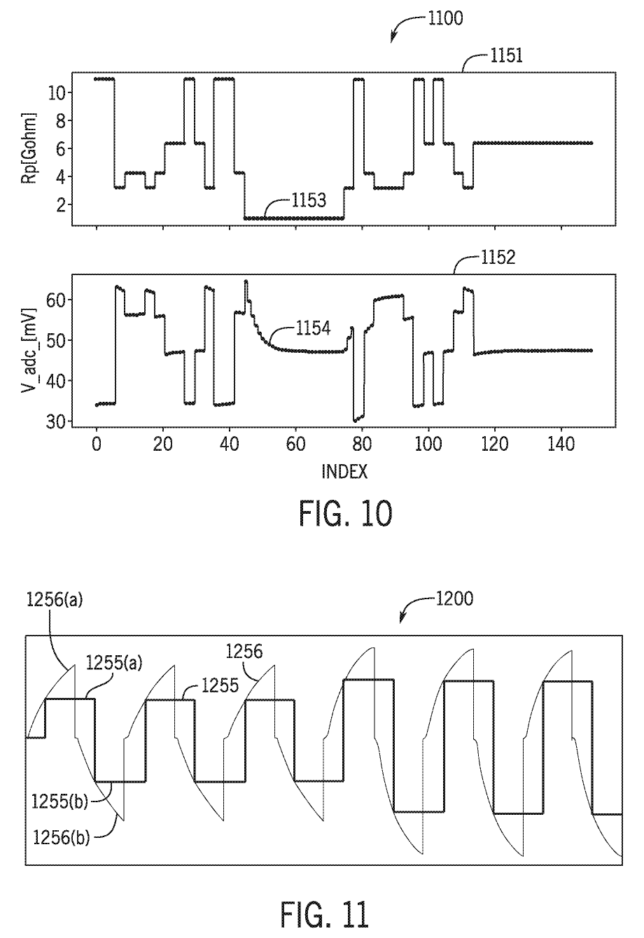

FIG. 10 depicts plots 1100 of pore resistance 1151 and output voltage 1152 for an example nanopore depicting hysteresis/memory effects, such as those caused by the longer time constant of certain embodiments. More specifically, at each operating point, the pore resistance 1151 depicts a constant value that corresponds to a particular nucleotide/reporter presently translocated with respect to the nanopore. As subsequent translocations introduce different nucleotides/reporters, the pore resistance changes, and it is this pore resistance (or a proxy therefor) that is measured to identify each nucleotide as the sequencing operation proceeds. Measured ADC voltages corresponding to these pore resistance values are depicted in plot 1152. As can be seen by comparing plots 1151 and 1152, each discrete value of pore resistance corresponds to an exponentially increasing or decreasing voltage determined by the circuit behaviors described above. This can mean that subsequent measurements of the same pore resistance (e.g., region 1153 where pore resistance remains constant over a number of indices or translocation cycles) can result in different voltage measurements over these indices as depicted by region 1154. This can occur, for example because of a sequence of repeated nucleotides/reporters. Even in regions where the change is less pronounced, it may be desirable to compensate in the measurements.

FIG. 11 depicts a bi-phase readout technique 1200 for mitigating at least some effects of the hysteresis/memory effects described above. Curve 1256 can correspond to the voltage at the trans electrode that is sampled to determine the pore resistance and thus the presently translocated nucleotide/reporter. In the illustrated example, alternating polarities of stimulus pulses are applied such that the exponential curves have alternating polarities. Pulses having alternating polarities may include a single positive pulse followed by a single negative pulse or can include a sequence of positive pulses (e.g., 2, 3, 4, 5, …, etc. positive pulses) followed by a sequence of negative pulses (e.g., 2, 3, 4, 5, …, etc. negative pulses). Rather than measuring a single peak value of this curve, such as positive peak 1256(a) or negative peak 1256(b) to determine pore resistance, a differential measurement depicted by curve 1255 can be used instead. More specifically, a positive value 1255(a) corresponding to a particular point on the rising portion of curve 1256 associated with a positive stimulus pulse and a negative value 1255(b) corresponding to a corresponding point on the declining portion of curve 1256 associated with a negative stimulus pulse can be used. The difference between these values, e.g., the peak-to-peak height of waveform 1255 between points 1255(a) and 1255(b) can be used to compute the pore resistance. Because the long time constant hysteresis effects are present in both the positive and negative values, the difference between them effectively cancels out this long term hysteresis effect, which can provide more accurate measurements that are not affected by prior stimulus or conditions of the nanopore. Additionally, this differential value provides a larger magnitude signal (e.g., approximately 2x), which can allow for easier measurements as well as potentially improving a signal to noise ratio of the measurement.

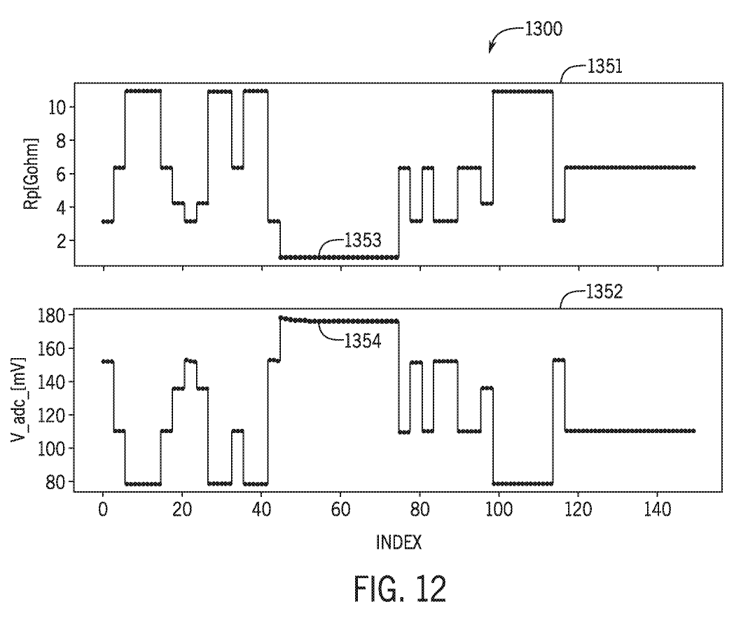

FIG. 12 depicts plots 1300 of pore resistance 1351 and output voltage 1352 for an example nanopore employing bi-phase readout as described above. The pore resistance values of curve 1351 are the same as those in curve 1151 described above. By using a bi-phase voltage measurement as described above with respect to FIG. 11, each measured voltage value corresponds more closely to the corresponding pore resistance because the hysteresis effects have been compensated.

### Example Nanopore Integrated Configuration and Control Structures

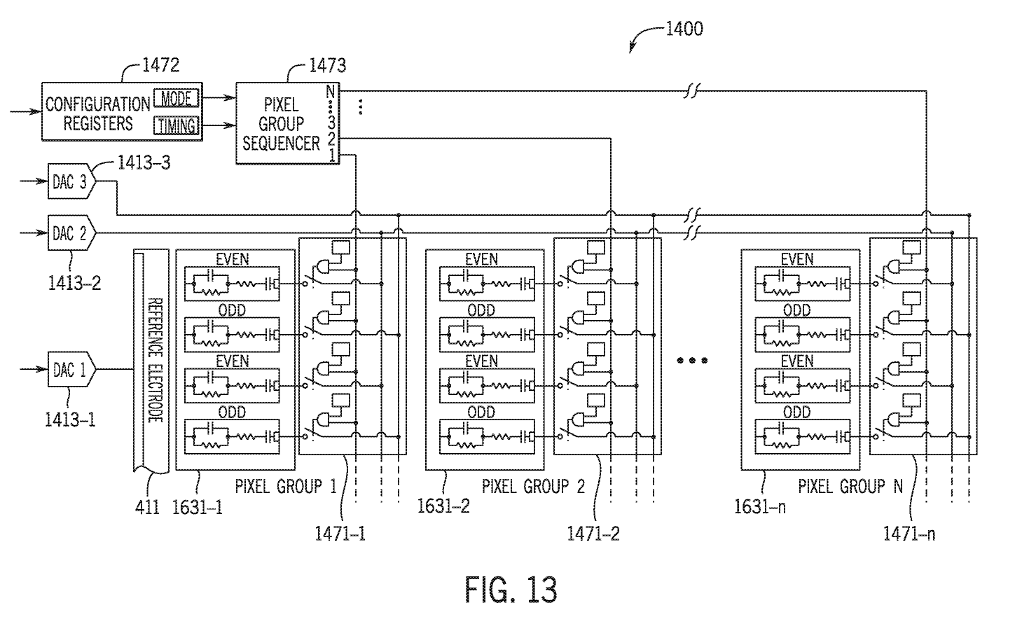

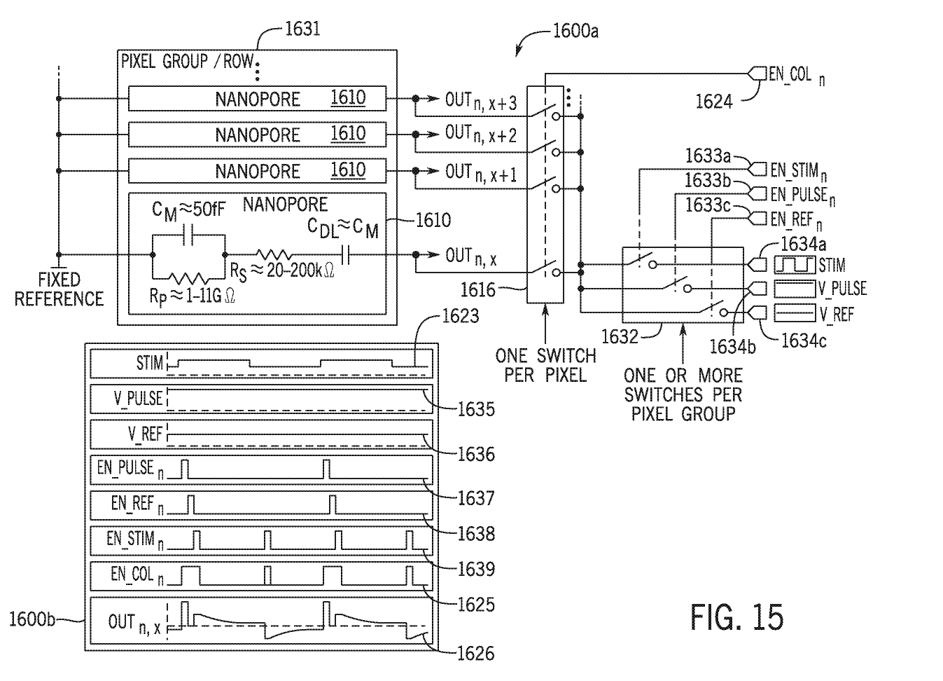

FIG. 13 is a simplified schematic 1400 of a nanopore integrated circuit with multiple nanopore groups driven from the ADC side/trans electrode. More specifically, the nanopore groups include a plurality of “Pixel Groups” 1, 2, …, N. Each pixel group can further include a series of “Sub-Groups” 1631-1, 1631-2, … 1631-n. The Pixel groups can, but need not, be column groups, and the Sub-Groups can be row groups, for example, corresponding to row group 1631 described below with reference to FIG. 15. Likewise, pixel groups can correspond to larger groupings, e.g., groups of sub-groups. The sub-groups may, but need not, correspond to a single linear row and could be multiple rows, portions of rows, and/or other geometric arrangements of nanopores/pixels. Similarly, the pixel groups may, but need not, correspond to a single linear column and could be multiple columns, portions of columns, and/or other geometric arrangements of the “sub-groups.” In short, the pixel groups and sub-groups can incorporate any suitable geometric arrangement of a plurality of nanopores.

Each of the nanopores can be coupled to a single common/cis electrode 411. Each pixel group an include pixel group control circuitry 1471-1, 1471-2, … 1471-n. This pixel group control circuitry is depicted in simplified form in FIG. 13 but can be as described below with reference to FIG. 15. That is each pixel group control circuitry block can include elements such as switching arrangements illustrated herein that can selectively enable or disable individual sub-groups, selectively apply stimulus pulses to individual sub-groups, and selectively couple measurement analog to digital converters (not shown). Also illustrated in FIG. 13 are digital to analog converters (“DACs”) 1413-1, 1413-2, and 1413-3 to allow different bias voltages (applied to the common/cis electrode 411 by DAC 1413-1) and/or stimulus voltages (applied to the ADC side/trans electrodes via DACs 1413-2 and/or DAC1413-3) to allow for different modes of operation with different voltage levels.

The ability to selectively apply stimulus pulses and/or take measurements to different nanopores/pixels with hierarchical groupings (e.g., selected pixel groups or sub-groups) can allow for simultaneous operation of various nanopores/pixels in different phases of operation without adverse interaction between nanopores/pixels using the principles described above. This can be achieved by overall control circuitry including configuration registers 1472 and pixel group sequencer 1473. Both of these elements can be implemented using an appropriate combination of logical circuit elements or blocks formed on the integrated circuit according to the requirements of a given semiconductor process. Some portions of this circuitry may be implemented with fixed logic, while other portions may be implemented with programmable logic.

As one example, configuration registers 1472 may include one or more registers specifying the mode(s) of various pixel groups and/or sub-groups, such as the stimulus mode (the nanopores are being stimulated), quiet mode (the nanopore voltages are decaying responsive to a particular reporter/nucleotide), or measurement mode (the output voltages are being sampled/measured). These registers may be programmed with value indicating particular modes and the associated timings. Configuration registers 1472 may also include timing registers specifying which pixel groups and/or sub-groups are being operated in the respective modes. These registers may be programmed with values indicating particular modes and the associated timings. Pixel group sequencer 1473 can be responsive to these configuration registers and can include logic to drive the respective pixel group control circuitry to accomplish the desired sequencing. In at least some implementations, the pixel group sequencer may also be programmable to allow for various operating sequences depending on the particular requirements of a given situation.

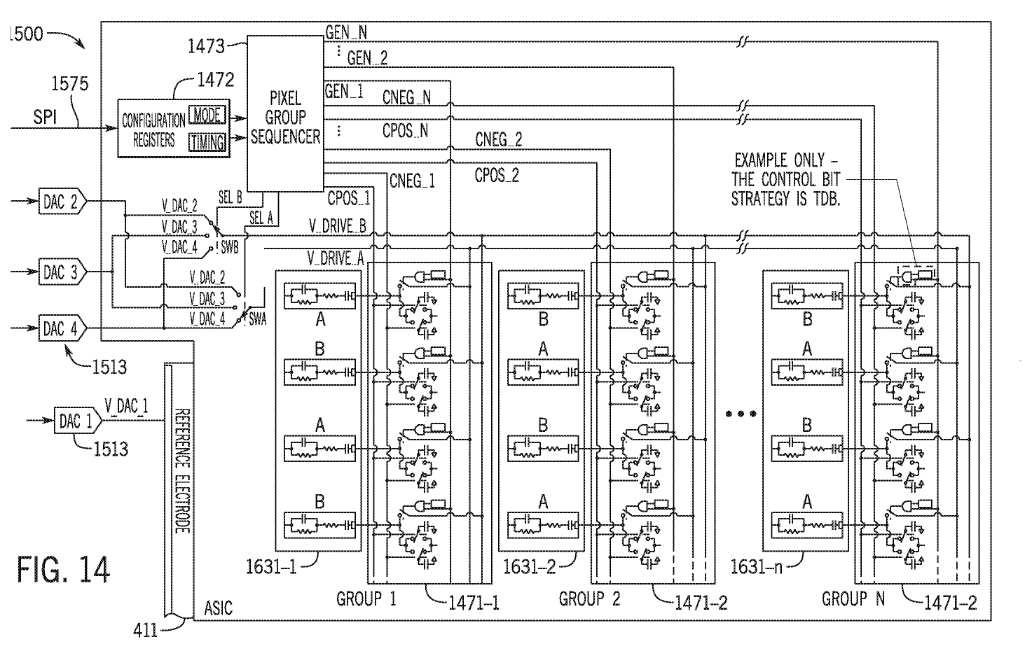

FIG. 14 is a simplified schematic of a nanopore integrated circuit 1500 with multiple nanopore groups 1631-1, 1631-2, … 1631-n driven from the ADC side/trans electrode. This schematic is similar to the schematic of FIG. 13, and items having corresponding reference numbers are as described above. In the schematic of FIG. 14, the depiction of pixel group control circuitry 1471-1, 1471-2, … 1471-n has been expanded to include further details such as the output capacitors and stimulus signal/output capacitor switching arrangements were described below with respect to FIG. 15. It should be noted that the control bit topology depicted in FIG. 14 is merely one example, and other circuitry variations for selective enable/disable control of nanopores/pixels could also be employed. Also depicted in FIG. 14 is a serial peripheral interface (SPI) 1575 allowing for control of the configuration registers and thereby pixel group sequencer 1473 from another devices, such as a microcontroller or microprocessor that is responsive to user input or otherwise operable to select the desired operating modes and timings as described above. Finally, FIG. 14 also depicts an expanded number of DACs 1513 as compared to FIG. 13, allowing for a greater range of voltages to be applied to the nanopores/pixels allowing for more operating modes. Coupling of these DACs 1513 to the respective nanopores/pixels can be controlled by switching arrangements driven by pixel group sequencer 1473 as described above.

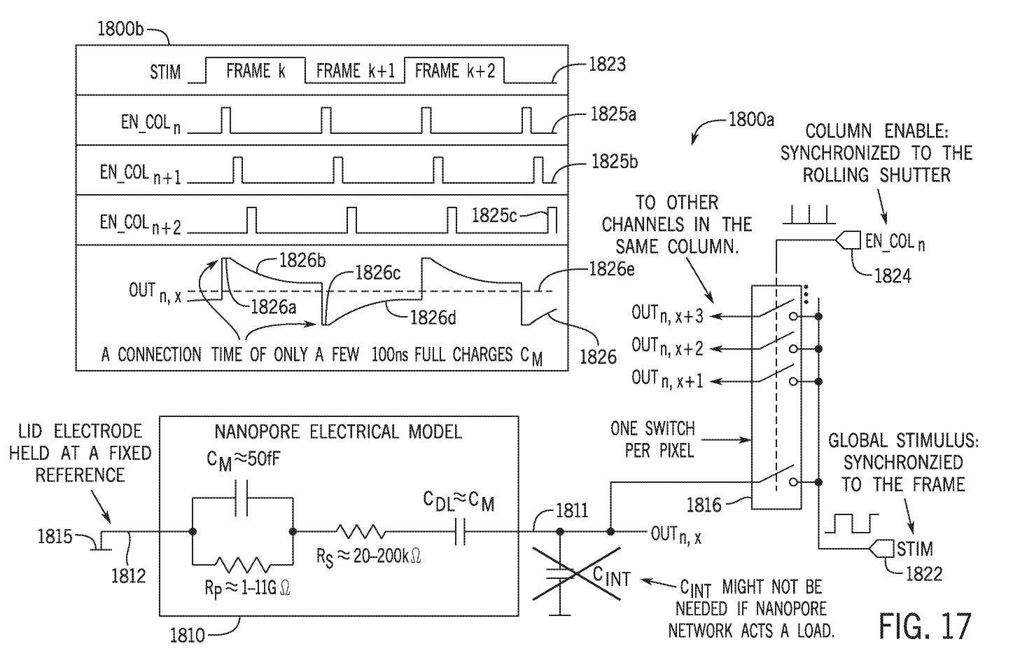

FIG. 15 illustrates various example operating waveforms 1600b of a nanopore driven from the ADC side/trans electrode together with additional details of the drive circuitry depicted in simplified schematic 1600a. Schematic 1600a depicts a pixel group or row 1631 of nanopores 1610. Nanopores/pixels 1610 can be, for example, nanopores 400a represented by equivalent circuit 400b (simplified for FIG. 15), as described above with reference to FIG. 4. Although described as a row in one example, the actual grouping arrangement of nanopores/pixels 1610 need not be a linear row, as other arrangement geometries such as a portion of a row, one or more columns or portions thereof, one or more rows plus one or more portions of rows, columns plus portions of columns, and groupings having other rectilinear or non-linear shapes could be employed as appropriate for a given application. Also depicted in schematic 1600a is switch 1616, which can includes a per-nanopore/pixel switch as described below with reference to FIG. 17, including terminal 1624 that can receive a column enable signal (similar to terminal 324 and corresponding column enable signal(s) described below with reference to FIG. 17) for selectively enabling nanopores/pixels 1610 of the group 1631.

Additionally, switches 1632 can include one or more switches per pixel group, allowing a variety of signals to be selectively delivered to per pixel switch(es) 1616 and thus provided to individual nanopores/pixels 1610 of the pixel group 1631. As one example, these can include a stimulus signal STIM/1623 (similar to the stimulus signal described above) applied to terminal 1634a, a V_PULSE signal 1635 applied to terminal 1634b, and a V_REF signal 1636 applied to terminal 1634c. These signals are also depicted in waveform plot 1600b, which includes waveforms similar to those described below with reference to FIG. 17. For example, stimulus waveform 1623 corresponding to stimulus waveform 1823, column enable signal 1625 corresponds to column enable signals 1825a–1825c, and output signal 1626 generally corresponds to output signal 1626. Also depicted in waveform plot 1600b are the V_PULSE signal 1635 and V_REF signal 1636. Finally, waveform plot 1600b depicts the EN_PULSEn signal 1637, EN_REFn signal 1638, and EN_STIMn signal 1639, which are the drive signals to per group switches 1632 to selectively couple the STIM, V_PULSE, and V_REF signals to the per-pixel switch(es) 1616 and thereby to the individual nanopores/pixels 1610.

The configuration depicted in FIG. 15 is merely one example implementation, and other specific per-pixel and per-group switch arrangements could be provided to provide the required stimulus signals as appropriate for any given implementation. To summarize FIG. 15, for controlling an individual nanopore/pixel 1610, it may be desirable, in at least some applications, to drive the nanopore/pixel ADC side/trans electrodes with other signals in addition to the stimulus signal 1623. As shown in FIG. 15, one or more per pixel group switches 1632 could be used to select from multiple signals (e.g., STIM/1623, V_PULSE/1635, and V_REF/1636) to drive the pixel group. The individual per-pixel switches 1616 can provide isolation while a measurement is being made. If the nanopores/pixels 1610 in a group 1631 need to be driven by a specific voltage, the pixel isolation switches 1616 can be closed. As depicted in waveform plot 1600b, more complex pixel control can be implemented. For example, the various control signals for each pixel group 1631 can be staggered in time to keep each pixel group synchronized to a rolling shutter. These signals can be generated outside the pixel array, so that no per-pixel bit storage is required.

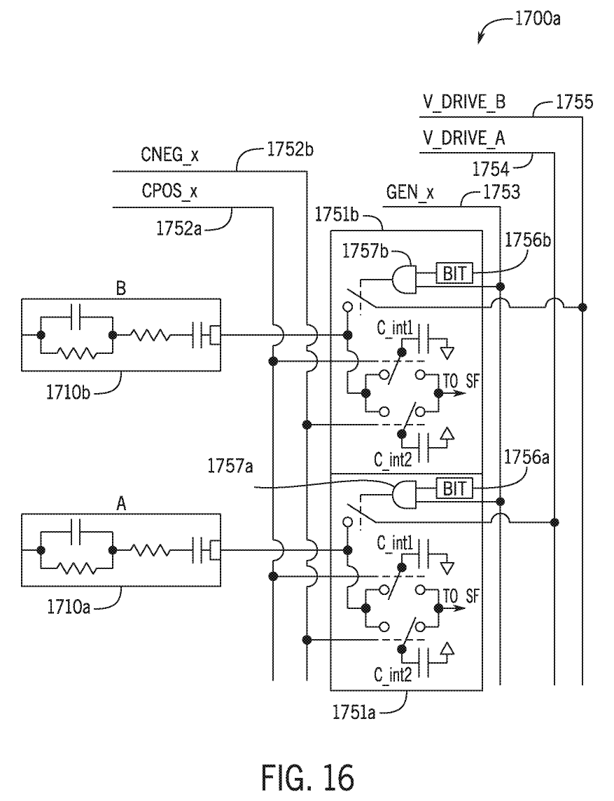

FIG. 16 illustrates a schematic diagram 1700a of circuitry for driving and reading separate groups of nanopores driven from the ADC side/trans electrode. Nanopore/pixel 1710a represents one or more nanopores/pixels to be driven in a first group “A,” and nanopore/pixel 1710b represents one or more nanopores/pixels to be driven in a second group “B.” Each of nanopores/pixels 1710a/1710b can be, for example, nanopores 400a represented by equivalent circuit 400b (simplified for FIG. 16). Each of nanopores/pixels 1710a/1710b can be selectively coupled to their respective drive signals by a switching arrangement 1751a/1751b. Switching arrangements 1751a/1751b can also selectively couple such the nanopores/pixels 1710a/1710b to respective, per-pixel (output) integrating capacitors C_int1 and C_int2, respectively corresponding to either a positive or negative stimulus pulse. These dual integrating capacitors can, for example, be as described above with respect to the universal pixel circuit described above with reference to FIG. 6.

Switching arrangements 1751a/1751b can each include multiple individual switching elements to accomplish the above-described selective couplings. For example, switching arrangement 1751a can include a first switching element that can selectively couple a stimulus signal from V_DRIVE_A bus 1754 to the trans electrode of nanopore/pixel 1710a responsive to a control signal from an AND gate 1757a that is itself responsive to the rolling shutter signal GEN_x (discussed in greater detail above with respect to FIG. 9A) and an enable or mask bit 1756a for selectively enabling or disabling a given nanopore/pixel. Switching arrangement 1751a can also include a pair of complementarily switched switching devices responsive respectively to the CPOS_x signal 1752a and the CNEG_x signal 1752b. These signals can toggle a connection of the ADC side/trans electrodes of nanopores/pixels 1710a/1710b to the respective (output) integrating capacitors C_int1 and C_int2 corresponding to positive or negative stimulus pulses applied via the respective drive busses.

FIG. 17 illustrates various operating waveforms 1800b of a nanopore driven from the ADC side/trans electrode together with additional details of the drive circuitry illustrated in simplified schematic 1800a. Per-pixel switch 1816 can include a plurality of switches, each switch coupled to a separate nanopore/pixel (although only a single nanopore/pixel 1810) is shown, which can correspond to, for example, nanopores 400a represented by equivalent circuit 400b (simplified for FIG. 17). These respective switches 1816 can be driven by respective column enable signals 1825a/1825b/1825c, coupled to terminal 1824.

A global stimulus rectangular wave 1823 can be synchronized to an ASIC frame (e.g., a single “image” taken by the ASIC), and the staggered COL_ENₙ pulse stream signals 1825a, 1825b, 1825c, etc. can control the timing for when the global stimulus is momentarily connected to a respective nanopore/pixel’s trans electrode, with the stimulus signal being applied to different pixels (or pixel groups) at staged times as part of a rolling stimulus operation or applied universally in a global stimulus operation. In other implementations, the stimulus signal need not be limited to per-frame synchronization as it could alternatively be varied/selected with sub-frame timing resolution for particular nanopore groups or sub-groups. Additionally, as described elsewhere herein (e.g., with respect to FIG. 14&15), the stimulus signal can be controlled and timed for respective pixel groups. This can provide flexibility allowing one pixel group to be driven by one voltage while other groups are driven by other voltages. In at least some implementations, a connection time on the order of a few hundred nanoseconds can be sufficient to fully charge membrane capacitance CM, although the specific timing can vary as appropriate for a given embodiment. Timing of the COL_ENₙ signals 1825a, 1825b, 1825c, etc. can be synchronized to a rolling shutter so that the output voltage for each pixel has settled in time for ADC readout of that nanopore/pixel. As illustrated by waveform 1826, in frame k, application of the positive phase of stimulus signal 1823 to a given nanopore/pixel 1610 by closing corresponding switch 1616 can result in the output voltage OUTn,x rising to the positive stimulus value, as depicted in waveform segment 1826a. Once the stimulus signal is de-coupled from the nanopore/pixel, by opening corresponding switch 1816, the output voltage 1826 begins an exponential decay 1826b determined by operation of the circuit of the nanopore/pixel. As noted above, different reporters translocated with respect to the nanopore will affect the nanopore resistance RP, and thus the rate at which the voltage decays and the resulting end voltage at the time of ADC readout, thereby allowing a specific reporter (and thus a corresponding nucleotide) to be identified. An example stimulus signal is described in more detail above with reference to FIG. 8.

As noted above, stimulus signal 1823 can alternate polarities between image frames. Two or more image frames can correspond to a single “loading” of the nanopore, allowing multiple readings to be correlated to improve measurement reliability and/or accuracy. As illustrated by waveforms 1800b, during frame k+1 the staggered COL_ENₙ pulse stream signals 1825a, 1825b, 1825c, etc. can also apply the negative stimulus signal to respective nanopores/pixels by closing corresponding switches 1816, resulting in “output” voltage 1826c appearing at the ADC side/trans electrode 1811. Thereafter, when the corresponding switch 1816 opens, the output voltage can decay, as depicted by decay segment 1826d, allowing for measurement by the ADC as described above, with the alternating polarities repeating for subsequent image frames k+2, etc.

Coupled noise as between adjacent nanopores 1810 can be reduced by using two complementary global rectangular waves 1823 (only one shown) and have consecutive pixel drive polarities/phases alternate such that a positive phase is applied to a given nanopore/pixel (group) and the negative phase is applied to one or more adjacent or nearby nanopore/pixel(s) (groups). The architecture illustrated in FIG. 17 can support more than one switch per nanopore/pixel and multiple global waveforms to provide additional control for alternative nanopore stimulus and sampling patterns, techniques or algorithms.

From the foregoing, it will be appreciated that a wide variety of implementations of the devices and techniques described herein are possible. Examples of these implementations can include:

Example 1. A nanopore integrated circuit comprising:
a semiconductor substrate;
a plurality of nanopore cells formed over the substrate, each nanopore cell havinga cis electrode connection and a trans electrode, wherein the plurality of nanopore cells are grouped into a plurality of nanopore pixel groups; drive circuitry including one or more switching devices formed on the substrate and coupled to the trans electrodes of the plurality of nanopore cells that: delivers stimulus signals to the trans electrodes of the plurality of nanopore cells, wherein the stimulus signals are delivered to the plurality of nanopore pixel groups in a rolling stimulus operation; and measures output signals of the plurality of nanopore pixel groups in a rolling shutter operation, wherein an output signal of a particular nanopore cell is produced by the nanopore cell in response to a stimulus signal and corresponds to an analyte translocated with respect to the particular nanopore cell.

Example 2. The nanopore integrated circuit of claim 1 wherein the stimulus signals include stimulus pulses of alternating polarities.

Example 3. The nanopore integrated circuit of claim 2 wherein the output signals are measured using a bi-phase measurement corresponding to the stimulus pulses of alternating polarities.

Example 4. The nanopore integrated circuit of claim 1 wherein the stimulus signals delivered to adjacent pixel groups include positive stimulus pulses delivered to a first subset of the adjacent pixel groups and negative stimulus pulses delivered to a second subset of the adjacent pixel groups.

Example 5. The nanopore integrated circuit of claim 1 wherein the drive circuitry delivers input stimulus signals during a crosstalk and settling time interval and measures the output signals during a quiet interval following the crosstalk and settling time interval.

Example 6. The nanopore integrated circuit of claim 1 wherein at least one of the cis electrode and the trans electrodes is a non-Faradaic electrode.

Example 7. The nanopore integrated circuit of claim 1 wherein the drive circuitry is further configured to deliver one or more electrical signals distinct from the stimulus signals to the plurality of nanopore cells to perform additional nanopore unit cell operations.

Example 8. The nanopore integrated circuit of claim 1 wherein:
the nanopore integrated circuit further comprises one or more analog to digital converters for measuring the output signals of the plurality of nanopore pixel groups; and
the rolling shutter operation measuring output signals of the plurality of nanopore pixel groups is coordinated with a readout timing of the one or more analog to digital converters.

Example 9. A method of driving a nanopore integrated circuit having a plurality of nanopore cells grouped into a plurality of nanopore pixel groups, each nanopore cell having a cis electrode connection and a trans electrode, the method comprising:
delivering stimulus signals to the trans electrodes of the plurality of nanopore cells, wherein the stimulus signals are delivered to the plurality of nanopore pixel groups in a rolling stimulus operation; and
measuring output signals of the plurality of nanopore pixel groups in a rolling shutter operation, wherein an output signal of a particular nanopore cell is produced by the nanopore cell in response to a stimulus signal and corresponds to an analyte translocated with respect to the particular nanopore cell.

Example 10. The method of claim 9 wherein the stimulus signals include stimulus pulses of alternating polarities.

Example 11. The method of claim 10 further comprising measuring the output signal using a bi-phase measurement corresponding to the stimulus pulses of alternating polarities.

Example 12. The method of claim 9 wherein the stimulus signals delivered to adjacent pixel groups include positive stimulus pulses delivered to a first subset of the adjacent pixel groups and negative stimulus pulses delivered to a second subset of the adjacent pixel groups.

Example 13. The method of claim 9 wherein:
delivering stimulus signals to the trans electrodes of the plurality of nanopore cells occurs during a crosstalk and settling time interval; and
measuring the output signals occurs during a quiet interval following the crosstalk and settling time interval.

Example 14. The method of claim 9 further comprising delivering one or more electrical signals distinct from the stimulus signals to the plurality of nanopore cells to perform additional nanopore unit cell operations.

Example 15. The method of claim 9 wherein the nanopore integrated circuit includes one or more analog to digital converters for measuring the output signals of the plurality of nanopore pixel groups and the method further comprises:
coordinating the rolling shutter operation measuring output signals of the plurality of nanopore pixel groups with a readout timing of the one or more analog to digital converters.

Example 16. A nanopore integrated circuit comprising:
a semiconductor substrate;
a plurality of nanopore cells formed over the substrate, each nanopore cell having a cis electrode connection and a trans electrode, wherein the plurality of nanopore cells are grouped into a plurality of nanopore pixel groups;
drive circuitry including one or more switching devices formed on the substrate and coupled to the trans electrodes of the plurality of nanopore cells that:
delivers stimulus signals to the trans electrodes of one or more nanopore pixel groups; and
performs a rolling shutter operation on the one or more nanopore pixel groups by selectively measuring output signals at the trans electrodes of nanopore cells of the one or more pixel groups, the output signals being produced by the nanopore cells in response to the stimulus signals, wherein an output signal of a particular nanopore cell corresponds to an analyte translocated with respect to the particular nanopore cell.

Example 17. The nanopore integrated circuit of claim 16 wherein the stimulus signals include stimulus pulses of alternating polarities.

Example 18. The nanopore integrated circuit of claim 17 wherein the output signals are measured using a bi-phase measurement corresponding to the stimulus pulses of alternating polarities.

Example 19. The nanopore integrated circuit of claim 16 wherein the stimulus signals delivered to adjacent pixel groups include positive stimulus pulses delivered to a first subset of the adjacent pixel groups and negative stimulus pulses delivered to a second subset of the adjacent pixel groups.

Example 20. The nanopore integrated circuit of claim 16 wherein the drive circuitry delivers input stimulus signals during a crosstalk and settling time interval and measures the output signals during a quiet interval following the crosstalk and settling time interval.

Example 21. The nanopore integrated circuit of claim 16 wherein:
the nanopore integrated circuit further comprises one or more analog to digital converters for measuring the output signals of the plurality of nanopore pixel groups; and
the rolling shutter operation measuring output signals of the plurality of nanopore pixel groups is coordinated with a readout timing of the one or more analog to digital converters.

Example 23. A method of driving a nanopore integrated circuit having a plurality of nanopore cells grouped into a plurality of nanopore pixel groups, each nanopore cell having a cis electrode connection and a trans electrode, the method comprising:
delivering stimulus signals to the trans electrodes of one or more nanopore pixel groups; and
performing a rolling shutter operation on the one or more nanopore pixel groups by selectively measuring output signals at the trans electrodes of nanopore cells of the one or more pixel groups, the output signals being produced by the nanopore cells in response to the stimulus signals, wherein an output signal of a particular nanopore cell corresponds to an analyte translocated with respect to the particular nanopore cell.

Example 24. The method of claim 23 wherein the stimulus signals include stimulus pulses of alternating polarities.

Example 25. The method of claim 24 further comprising measuring the output signals using a bi-phase measurement corresponding to the stimulus pulses of alternating polarities.

Example 26. The method of claim 23 wherein the stimulus signals delivered to adjacent pixel groups include positive stimulus pulses delivered to a first subset of the adjacent pixel groups and negative stimulus pulses delivered to a second subset of the adjacent pixel groups.

Example 27. The method of claim 23 wherein:
delivering stimulus signals to the trans electrodes of the plurality of nanopore cells occurs during a crosstalk and settling time interval; and
measuring the output signals occurs during a quiet interval following the crosstalk and settling time interval.

Example 28. The method of claim 23 wherein the nanopore integrated circuit includes one or more analog to digital converters for measuring the output signals of the plurality of nanopore pixel groups and the method further comprises:
coordinating the rolling shutter operation measuring output signals of the plurality of nanopore pixel groups with a readout timing of the one or more analog to digital converters.

Example 29. A nanopore integrated circuit comprising:
a semiconductor substrate;
a plurality of nanopore cells formed over the substrate, each nanopore cell having a cis electrode connection and a trans electrode, wherein the plurality of nanopore cells are grouped into a plurality of nanopore pixel groups;
drive circuitry including one or more switching devices formed on the substrate and coupled to the trans electrodes of the plurality of nanopore cells that:
performs a rolling stimulus operation to deliver stimulus signals to the trans electrodes of the plurality of nanopore cells, wherein the stimulus signals are delivered to one or more nanopore pixel groups; and
measures output signals of the one or more nanopore pixel groups, wherein an output signal of a particular nanopore cell is produced by the nanopore cell in response to a stimulus signal and corresponds to an analyte translocated with respect to the particular nanopore cell.

Example 30. The nanopore integrated circuit of claim 29 wherein the stimulus signals include stimulus pulses of alternating polarities.

Example 31. The nanopore integrated circuit of claim 30 wherein the output signals are measured using a bi-phase measurement corresponding to the stimulus pulses of alternating polarities.

Example 32. The nanopore integrated circuit of claim 29 wherein the stimulus signals delivered to adjacent pixel groups include positive stimulus pulses delivered to a first subset of the adjacent pixel groups and negative stimulus pulses delivered to a second subset of the adjacent pixel groups.

Example 33. The nanopore integrated circuit of claim 29 wherein the drive circuitry delivers input stimulus signals during a crosstalk and settling time interval and measures the output signals during a quiet interval following the crosstalk and settling time interval.

Example 34. A method of driving a nanopore integrated circuit having a plurality of nanopore cells grouped into a plurality of nanopore pixel groups, each nanopore cell having a cis electrode connection and a trans electrode, the method comprising:
performing a rolling stimulus operation to deliver stimulus signals to the trans electrodes of one or more nanopore pixel groups; and
measuring output signals of the one or more of nanopore pixel groups, wherein an output signal of a particular nanopore cell is produced by the nanopore cell in response to a stimulus signal and corresponds to an analyte translocated with respect to the particular nanopore cell.

Example 35. The method of claim 34 wherein the stimulus signals include stimulus pulses of alternating polarities.

Example 36. The method of claim 35 further comprising measuring the output signals using a bi-phase measurement corresponding to the stimulus pulses of alternating polarities.

Example 37. The method of claim 34 wherein the stimulus signals delivered to adjacent pixel groups include positive stimulus pulses delivered to a first subset of the adjacent pixel groups and negative stimulus pulses delivered to a second subset of the adjacent pixel groups.

Example 38. The method of claim 34 wherein:
delivering stimulus signals to the trans electrodes of the plurality of nanopore cells occurs during a crosstalk and settling time interval; and
measuring the output signals occurs during a quiet interval following the crosstalk and settling time interval.

In certain embodiments, the nanopore sequencing system and/or methods may incorporate certain features of U.S. Patent Publications US20250242348A1, US20250242348A1, US20250019758A1, US20250109433A1, or U.S. Patent No. 11473067B2, the disclosure of which are incorporated by reference herein in their entireties.

### Additional Notes

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

Reference throughout the specification to “one example”, “another example”, “an example”, and so forth, means that a particular element (e.g., feature, structure, and/or characteristic) described in connection with the example is included in at least one example described herein, and may or may not be present in other examples. In addition, it is to be understood that the described elements for any example may be combined in any suitable manner in the various examples unless the context clearly dictates otherwise.

It is to be understood that the ranges provided herein include the stated range and any value or sub-range within the stated range, as if such value or sub-range were explicitly recited. For example, a range from about 2 nm to about 20 nm should be interpreted to include not only the explicitly recited limits of from about 2 nm to about 20 nm, but also to include individual values, such as about 3.5 nm, about 8 nm, about 18.2 nm, etc., and sub-ranges, such as from about 5 nm to about 10 nm, etc. Furthermore, when “about” and/or “substantially” are/is utilized to describe a value, this is meant to encompass minor variations (e.g., up to +/−10%) from the stated value.

While several examples have been described in detail, it is to be understood that the disclosed examples may be modified. Therefore, the foregoing description is to be considered non-limiting.

While certain examples have been described, these examples have been presented by way of example only and are not intended to limit the scope of the disclosure. Indeed, the novel methods and systems described herein may be embodied in a variety of other forms.

Furthermore, various omissions, substitutions and changes in the systems and methods described herein may be made without departing from the spirit of the disclosure. The accompanying claims and their equivalents are intended to cover such forms or modifications as would fall within the scope and spirit of the disclosure.

Features, materials, characteristics, or groups described in conjunction with a particular aspect, or example are to be understood to be applicable to any other aspect or example described in this section or elsewhere in this specification unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The protection is not restricted to the details of any foregoing examples. The protection extends to any novel one, or any novel combination, of the features disclosed in this specification (including any accompanying claims, abstract and drawings), or to any novel one, or any novel combination, of the steps of any method or process so disclosed.

Furthermore, certain features that are described in this disclosure in the context of separate implementations can also be implemented in combination in a single implementation. Conversely, various features that are described in the context of a single implementation can also be implemented in multiple implementations separately or in any suitable sub-combination. Moreover, although features may be described above as acting in certain combinations, one or more features from a claimed combination can, in some cases, be excised from the combination, and the combination may be claimed as a sub-combination or variation of a sub-combination.

Moreover, while operations may be depicted in the drawings or described in the specification in a particular order, such operations need not be performed in the particular order shown or in sequential order, or that all operations be performed, to achieve desirable results. Other operations that are not depicted or described can be incorporated in the example methods and processes. For example, one or more additional operations can be performed before, after, simultaneously, or between any of the described operations. Further, the operations may be rearranged or reordered in other implementations. Those skilled in the art will appreciate that in some examples, the actual steps taken in the processes illustrated and/or disclosed may differ from those shown in the figures. Depending on the example, certain of the steps described above may be removed or others may be added. Furthermore, the features and attributes of the specific examples disclosed above may be combined in different ways to form additional examples, all of which fall within the scope of the present disclosure. Also, the separation of various system components in the implementations described above should not be understood as requiring such separation in all implementations, and it should be understood that the described components and systems can generally be integrated together in a single product or packaged into multiple products.

For purposes of this disclosure, certain aspects, advantages, and novel features are described herein. Not necessarily all such advantages may be achieved in accordance with any particular example. Thus, for example, those skilled in the art will recognize that the disclosure may be embodied or carried out in a manner that achieves one advantage or a group of advantages as taught herein without necessarily achieving other advantages as may be taught or suggested herein.

Conditional language, such as “can,” “could,” “might,” or “may,” unless specifically stated otherwise, or otherwise understood within the context as used, is generally intended to convey that certain examples include, while other examples do not include, certain features, elements, and/or steps. Thus, such conditional language is not generally intended to imply that features, elements, and/or steps are in any way required for one or more examples or that one or more examples necessarily include logic for deciding, with or without user input or prompting, whether these features, elements, and/or steps are included or are to be performed in any particular example.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain examples require the presence of at least one of X, at least one of Y, and at least one of Z.

Language of degree used herein, such as the terms “approximately,” “about,” “generally,” and “substantially” represent a value, amount, or characteristic close to the stated value, amount, or characteristic that still performs a desired function or achieves a desired result.

The scope of the present disclosure is not intended to be limited by the specific disclosures of preferred examples in this section or elsewhere in this specification and may be defined by claims as presented in this section or elsewhere in this specification or as presented in the future. The language of the claims is to be interpreted broadly based on the language employed in the claims and not limited to the examples described in the present specification or during the prosecution of the application, which examples are to be construed as non-exclusive.

Although the foregoing invention has been described in terms of certain preferred examples, other examples will be apparent to those of ordinary skill in the art. Additionally, other combinations, omissions, substitutions and modification will be apparent to the skilled artisan, in view of the disclosure herein. Accordingly, the present invention is not intended to be limited by the recitation of the preferred examples but is instead to be defined by reference to the appended claims. All references cited herein are incorporated by reference in their entirety.

The terminology used in the description presented herein is not intended to be interpreted in any limited or restrictive manner and unless otherwise indicated may refer to the ordinary meaning as would be understood by one of ordinary skill in the art in view of the specification. Furthermore, examples may comprise, consist of, consist essentially of, several novel features, no single one of which is solely responsible for its desirable attributes or is believed to be essential to practicing the examples herein described. As used herein, the section headings are for organizational purposes only and are not to be construed as limiting the described subject matter in any way. All literature and similar materials cited in this application, including but not limited to, patents, patent applications, articles, books, treatises, and internet web pages are expressly incorporated by reference in their entirety for any purpose. When definitions of terms in incorporated references appear to differ from the definitions provided in the present teachings, the definition provided in the present teachings shall control. It will be appreciated that there is an implied “about” prior to the temperatures, concentrations, times, etc. discussed in the present teachings, such that slight and insubstantial deviations are within the scope of the present teachings herein.

Although this disclosure is in the context of certain examples and examples, those of ordinary skill in the art will understand that the present disclosure extends beyond the specifically disclosed examples to other alternative examples and/or uses of the examples and obvious modifications and equivalents thereof. In addition, while several variations of the examples have been shown and described in detail, other modifications, which are within the scope of this disclosure, will be readily apparent to those of ordinary skill in the art based upon this disclosure. It is also contemplated that various combinations or sub-combinations of the specific features and aspects of the examples may be made and still fall within the scope of the disclosure. It should be understood that various features and aspects of the disclosed examples can be combined with, or substituted for, one another in order to form varying modes or examples of the disclosure. Thus, it is intended that the scope of the present disclosure herein disclosed should not be limited by the particular disclosed examples described above.

1. A nanopore integrated circuit comprising:
a semiconductor substrate;
a plurality of nanopore cells formed over the substrate, each nanopore cell having a cis electrode connection and a trans electrode, wherein the plurality of nanopore cells are grouped into a plurality of nanopore pixel groups;
drive circuitry including one or more switching devices formed on the substrate and coupled to the trans electrodes of the plurality of nanopore cells that:
delivers stimulus signals to the trans electrodes of the plurality of nanopore cells, wherein the stimulus signals are delivered to the plurality of nanopore pixel groups in a rolling stimulus operation; and
measures output signals of the plurality of nanopore pixel groups in a rolling shutter operation, wherein an output signal of a particular nanopore cell is produced by the nanopore cell in response to a stimulus signal and corresponds to an analyte translocated with respect to the particular nanopore cell.

2. The nanopore integrated circuit of claim 1 wherein the stimulus signals include stimulus pulses of alternating polarities.

3. The nanopore integrated circuit of claim 2 wherein the output signals are measured using a bi-phase measurement corresponding to the stimulus pulses of alternating polarities.

4. The nanopore integrated circuit of claim 1 wherein the stimulus signals delivered to adjacent pixel groups include positive stimulus pulses delivered to a first subset of the adjacent pixel groups and negative stimulus pulses delivered to a second subset of the adjacent pixel groups.

5. The nanopore integrated circuit of claim 1 wherein the drive circuitry delivers input stimulus signals during a crosstalk and settling time interval and measures the output signals during a quiet interval following the crosstalk and settling time interval.

6. The nanopore integrated circuit of claim 1 wherein at least one of the cis electrode and the trans electrodes is a non-Faradaic electrode.

7. The nanopore integrated circuit of claim 1 wherein the drive circuitry is further configured to deliver one or more electrical signals distinct from the stimulus signals to the plurality of nanopore cells to perform additional nanopore unit cell operations.

8. The nanopore integrated circuit of claim 1 wherein:
the nanopore integrated circuit further comprises one or more analog to digital converters for measuring the output signals of the plurality of nanopore pixel groups; and
the rolling shutter operation measuring output signals of the plurality of nanopore pixel groups is coordinated with a readout timing of the one or more analog to digital converters.

9. A method of driving a nanopore integrated circuit having a plurality of nanopore cells grouped into a plurality of nanopore pixel groups, each nanopore cell having a cis electrode connection and a trans electrode, the method comprising:
delivering stimulus signals to the trans electrodes of the plurality of nanopore cells, wherein the stimulus signals are delivered to the plurality of nanopore pixel groups in a rolling stimulus operation; and
measuring output signals of the plurality of nanopore pixel groups in a rolling shutter operation, wherein an output signal of a particular nanopore cell is produced by the nanopore cell in response to a stimulus signal and corresponds to an analyte translocated with respect to the particular nanopore cell.

10. The method of claim 9 wherein the stimulus signals include stimulus pulses of alternating polarities.

11. The method of claim 10 further comprising measuring the output signal using a bi-phase measurement corresponding to the stimulus pulses of alternating polarities.

12. The method of claim 9 wherein the stimulus signals delivered to adjacent pixel groups include positive stimulus pulses delivered to a first subset of the adjacent pixel groups and negative stimulus pulses delivered to a second subset of the adjacent pixel groups.

13. The method of claim 9 wherein:
delivering stimulus signals to the trans electrodes of the plurality of nanopore cells occurs during a crosstalk and settling time interval; and
measuring the output signals occurs during a quiet interval following the crosstalk and settling time interval.

14. The method of claim 9 further comprising delivering one or more electrical signals distinct from the stimulus signals to the plurality of nanopore cells to perform additional nanopore unit cell operations.

15. The method of claim 9 wherein the nanopore integrated circuit includes one or more analog to digital converters for measuring the output signals of the plurality of nanopore pixel groups and the method further comprises:
coordinating the rolling shutter operation measuring output signals of the plurality of nanopore pixel groups with a readout timing of the one or more analog to digital converters.

16. A nanopore integrated circuit comprising:
a semiconductor substrate;
a plurality of nanopore cells formed over the substrate, each nanopore cell having a cis electrode connection and a trans electrode, wherein the plurality of nanopore cells are grouped into a plurality of nanopore pixel groups;
drive circuitry including one or more switching devices formed on the substrate and coupled to the trans electrodes of the plurality of nanopore cells that:
delivers stimulus signals to the trans electrodes of one or more nanopore pixel groups; and
performs a rolling shutter operation on the one or more nanopore pixel groups by selectively measuring output signals at the trans electrodes of nanopore cells of the one or more pixel groups, the output signals being produced by the nanopore cells in response to the stimulus signals, wherein an output signal of a particular nanopore cell corresponds to an analyte translocated with respect to the particular nanopore cell.

17. The nanopore integrated circuit of claim 16 wherein the stimulus signals include stimulus pulses of alternating polarities.

18. The nanopore integrated circuit of claim 17 wherein the output signals are measured using a bi-phase measurement corresponding to the stimulus pulses of alternating polarities.

19. The nanopore integrated circuit of claim 16 wherein the stimulus signals delivered to adjacent pixel groups include positive stimulus pulses delivered to a first subset of the adjacent pixel groups and negative stimulus pulses delivered to a second subset of the adjacent pixel groups.

20. The nanopore integrated circuit of claim 16 wherein the drive circuitry delivers input stimulus signals during a crosstalk and settling time interval and measures the output signals during a quiet interval following the crosstalk and settling time interval.

21. The nanopore integrated circuit of claim 16 wherein:
the nanopore integrated circuit further comprises one or more analog to digital converters for measuring the output signals of the plurality of nanopore pixel groups; and
the rolling shutter operation measuring output signals of the plurality of nanopore pixel groups is coordinated with a readout timing of the one or more analog to digital converters.

22. A method of driving a nanopore integrated circuit having a plurality of nanopore cells grouped into a plurality of nanopore pixel groups, each nanopore cell having a cis electrode connection and a trans electrode, the method comprising:
delivering stimulus signals to the trans electrodes of one or more nanopore pixel groups; and
performing a rolling shutter operation on the one or more nanopore pixel groups by selectively measuring output signals at the trans electrodes of nanopore cells of the one or more pixel groups, the output signals being produced by the nanopore cells in response to the stimulus signals, wherein an output signal of a particular nanopore cell corresponds to an analyte translocated with respect to the particular nanopore cell.

23. The method of claim 22 wherein the stimulus signals include stimulus pulses of alternating polarities.

24. The method of claim 23 further comprising measuring the output signals using a bi-phase measurement corresponding to the stimulus pulses of alternating polarities.

25. The method of claim 22 wherein the stimulus signals delivered to adjacent pixel groups include positive stimulus pulses delivered to a first subset of the adjacent pixel groups and negative stimulus pulses delivered to a second subset of the adjacent pixel groups.

26. The method of claim 22 wherein:
delivering stimulus signals to the trans electrodes of the plurality of nanopore cells occurs during a crosstalk and settling time interval; and
measuring the output signals occurs during a quiet interval following the crosstalk and settling time interval.

27. The method of claim 22 wherein the nanopore integrated circuit includes one or more analog to digital converters for measuring the output signals of the plurality of nanopore pixel groups and the method further comprises:
coordinating the rolling shutter operation measuring output signals of the plurality of nanopore pixel groups with a readout timing of the one or more analog to digital converters.

28. A nanopore integrated circuit comprising:
a semiconductor substrate;
a plurality of nanopore cells formed over the substrate, each nanopore cell having a cis electrode connection and a trans electrode, wherein the plurality of nanopore cells are grouped into a plurality of nanopore pixel groups;
drive circuitry including one or more switching devices formed on the substrate and coupled to the trans electrodes of the plurality of nanopore cells that:
performs a rolling stimulus operation to deliver stimulus signals to the trans electrodes of the plurality of nanopore cells, wherein the stimulus signals are delivered to one or more nanopore pixel groups; and
measures output signals of the one or more nanopore pixel groups, wherein an output signal of a particular nanopore cell is produced by the nanopore cell in response to a stimulus signal and corresponds to an analyte translocated with respect to the particular nanopore cell.

29. The nanopore integrated circuit of claim 28 wherein the stimulus signals include stimulus pulses of alternating polarities.

30. The nanopore integrated circuit of claim 29 wherein the output signals are measured using a bi-phase measurement corresponding to the stimulus pulses of alternating polarities.

31. The nanopore integrated circuit of claim 28 wherein the stimulus signals delivered to adjacent pixel groups include positive stimulus pulses delivered to a first subset of the adjacent pixel groups and negative stimulus pulses delivered to a second subset of the adjacent pixel groups.

32. The nanopore integrated circuit of claim 28 wherein the drive circuitry delivers input stimulus signals during a crosstalk and settling time interval and measures the output signals during a quiet interval following the crosstalk and settling time interval.

33. A method of driving a nanopore integrated circuit having a plurality of nanopore cells grouped into a plurality of nanopore pixel groups, each nanopore cell having a cis electrode connection and a trans electrode, the method comprising:
performing a rolling stimulus operation to deliver stimulus signals to the trans electrodes of one or more nanopore pixel groups; and
measuring output signals of the one or more of nanopore pixel groups, wherein an output signal of a particular nanopore cell is produced by the nanopore cell in response to a stimulus signal and corresponds to an analyte translocated with respect to the particular nanopore cell.

34. The method of claim 33 wherein the stimulus signals include stimulus pulses of alternating polarities.

35. The method of claim 34 further comprising measuring the output signals using a bi-phase measurement corresponding to the stimulus pulses of alternating polarities.

36. The method of claim 33 wherein the stimulus signals delivered to adjacent pixel groups include positive stimulus pulses delivered to a first subset of the adjacent pixel groups and negative stimulus pulses delivered to a second subset of the adjacent pixel groups.

37. The method of claim 33 wherein:
delivering stimulus signals to the trans electrodes of the plurality of nanopore cells occurs during a crosstalk and settling time interval; and
measuring the output signals occurs during a quiet interval following the crosstalk and settling time interval.
